(19) United States
(12) Patent Application Publication
(10) Pub. No.: US 2023/0348987 A1
(43) Pub. Date: Nov. 2, 2023

(54) METHODS AND COMPOSITIONS FOR TREATING CANCER

(71) Applicant: PROMETTRE RESEARCH PTY LTD, South Melbourne, Victoria (AU)

(72) Inventors: Chau Hoang NGUYEN, South Melbourne (AU); John Timothy PRICE, South Melbourne (AU); Jeremy DRAKE, South Melbourne (AU)

(73) Assignee: PROMETTRE CANCER RESEARCH CO LTD, South Melbourne, Victoria (AU)

(21) Appl. No.: 18/006,284

(22) PCT Filed: Jul. 21, 2021
(86) PCT No.: PCT/AU2021/050785

§ 371 Date: Jan. 20, 2023

(30) Foreign Application Priority Data
Jul. 21, 2020 (AU) 2020902532

(51) Int. Cl. C12Q 1/6886; G01N 33/574; A61P 35/00; C12N 15/113; A61K 31/506; A61K 31/517; A61K 31/5377

(52) U.S. Cl. CPC C12Q 1/6886; G01N 33/57415; A61P 35/00; C12N 15/1138; A61K 31/506; A61K 31/517; A61K 31/5377; C12Q 2600/106; C12Q 2600/158; G01N 2333/71; C12N 2310/14; C12N 2310/531

(57) ABSTRACT

A method of predicting the responsiveness of a subject with cancer to treatment with a modulator of the HER signalling pathway, wherein the method comprises detecting the level of expression of EGR4-S in a sample of the subject, wherein the subject is likely to be responsive to treatment with the modulator of the HER signalling pathway if EGR4-S expression is at or above a predetermined level in the sample of the subject, and wherein the subject is likely to be resistant to treatment with the modulator of the HER signalling pathway if EGR4-S expression is at or below a predetermined level in the sample of the subject.

### FIELD OF THE INVENTION

The present invention relates to method of predicting the responsiveness of a subject with cancer to treatment with a modulator of the HER signalling pathway.

### BACKGROUND

Cancer is the second leading cause of death globally after cardiac disease. The World Health Organisation recently estimated that 8.2 million people die from cancer annually, with the most commonly diagnosed types of cancer being lung and breast (Ferlay et al., 2012). A frequently altered cell signalling pathway that is common to many types of cancer is the Human Epidermal growth factor Receptor (HER) pathway. Changes to components of this pathway that control cell proliferation have been demonstrated in many types of cancer. Breast cancers with this altered pathway (“HER2 activated”) represent approximately 20-25% of the total number of cases diagnosed annually (Slamon et al, 1987; Slamon et al., 1989). Between 80-90% of lung cancers have also been observed to have this altered (“HER1 activated”) pathway (Lynch et al, 2004; Al Olayan et al., 2012).

Historically, treatment of solid cancers has involved surgery, radiotherapy and/or chemotherapy. More recently, as researchers have come to understand the cellular changes that promote cancer growth, targeted therapies have been developed that specifically target and arrest these cancer-promoting cellular changes. Since components of the HER pathway are so frequently altered in cancer cells, several targeted drug therapies have been developed to improve patient prognosis. One class of these drugs, Tyrosine Kinase Inhibitors (TKIs), are effective in treating cancers like lung and breast that have activated HER signalling pathways. TKI drugs such as Lapatinib, Gefintinib and Erlotinib are currently used as a component in treating people that develop HER+ breast cancers or HER+ lung cancers because of their prognostic benefits (Antonicelli et al., 2013; Cameron et al., 2008).

Unfortunately, as with many cancer therapies, the use of HER pathway targeted drugs is limited by the problem of resistance—some HER+ individuals are not responsive to them and others acquire resistance to the drugs over time. Understanding the phenomenon of drug resistance is an important area of investigation as this will lead to improved treatment outcomes for all patients with HER pathway-activated cancers.

There is a need for methods of predicting the responsiveness of subjects with cancer to treatment with modulators of the HER signalling pathway.

### BRIEF DESCRIPTION OF THE DRAWINGS

FIG. 1: Oncogenic Transformation of Breast Cells Reduces their Response to Molecular Stress

(A) Protein expression in different types of MCF10a ‘normal’ breast cells transfected with an empty, control virus (mCherry) vs H-Ras. Higher HSP levels are seen with increased HSF1 expression. (B) Light microscopy of MCF10a cells shows control cells have an epithelial-like morphology where as oncogenically transformed cells have a mesenchymal morphology (C) Glycolytic capacity of oncogenically transformed MCF10a cells is significantly higher than non-transformed cells (***P<0.001). Upregulating HSF1 in oncogenically transformed cells results in significantly reduced glycolysis (**P<0.01). (D) Venn Diagram showing HSF1 effect on altered gene expression in oncogenically-transformed and non-transformed cells. EGR4 is the only gene regulated by HSF1 in both the normal and oncogenically transformed cells (E) EGR4-S protein expression is increased with HSF1 in the normal breast cells but decreased with HSF1 in oncogenically transformed breast cells.

FIG. 2: EGR4-S Expression is Upregulated in HER2/HER1 (EGFR)-Positive Breast Cancer Cells and is Correlated to HER2 Positive Cancer Relapse-Free Survival

(A) Protein expression in a panel of breast cancer cell lines of different sub-types. EGR4-S expression is higher in HER2+ cancer cell lines and in conjunction with reduced HSF1 expression; FIG. 2E shows a different version of this panel with molecular weight marker shown indicating ERG4-S expression. (B-D) Kaplan-Meier plots of patient survival over time. In HER2+ cancers, patients with high EGR4-S expression have significantly better survival (P=0.0025). In Basal cancers, EGR4-S expression is also linked to better survival although the separation between patient groups is not as clear (P=0.029). (E) EGR4-S expression is higher in HER2+ cancer cell lines and in conjunction with reduced HSF1 expression.

FIG. 3: EGR4-S Expression is Regulated by TKI Drug Treatment

(A) Active components of the HER signalling pathway (pHER2, pAKT) decrease in direct response to increasing concentrations of TKI drug treatment. EGR4-S expression is reduced as the components of the signalling pathway are reduced. (B) Suppression of EGR4-S expression with Lapatinib treatment is maintained over time in different types of HER2+ breast cancer cells. (C) TKI drugs have the same inhibitory effect on active components of the HER signalling pathway in Basal breast cancer cells with activated HER signalling. The decline in EGR4-S is present but not marked as the HER2+ cells.

FIG. 4: EGR4-S Expression is Regulated by Molecular Stress

(A) Increased HSF1 expression in HER2+ breast cancer cells resulted in greater HSP105 expression and reduction in EGR4-S (B) Increased HSF1 expression in Luminal breast cancer cells also resulted in greater HSP105 and reduction in EGR4-S (C) Knockdown of HSF1 resulted in a reduction in HSP90 and an increase in EGR4-S (D) Increasing HSF1 expression in HER2+ cells resulted in lower activated HER2 (pHER2) but had no effect on total HER2 levels (E) Higher concentrations of stress-inducing compound AUY922 increased expression of HSP105 but decreased expression of pHER2, total HER2 and EGR4-S. A similar pattern was identified with Sulfurophane treatment, with greater treatment concentrations causing increased HSP70 but reductions in pHER2 and EGR-S.

FIG. 5: Stress Enhances Metastatic Potential of Breast Cancer Cells

(A) Increased HSF1 in SkBr3 (HER2+) breast cancer cells resulted in significantly slower growth rate over time, (B) SkBr3 cells with high HSF1 have a more disorganised growth pattern when grown in 3D culture from single cell origin which is characteristic of more metastatic cancer types, (C) High HSF1 levels resulted in significantly more migratory behaviour (WT: **P<0.01, RDT: ***P<001) compared to control cancer cells (D) Knockdown of EGR4-S using shRNA in SkBr3 cells was confirmed with Western blot (E) SkBr3 cells with EGR4-S knockdown had slower growth over time compared to control cells.

FIG. 6: Molecular Stress (HSF1 Expression) in Breast Cancer Cells Makes them More Migratory but has No Effect on Normal Breast Cells

(A) HSF1 upregulation (HSF1 WT and ΔRDT) in MCF10a ‘normal’ breast cells resulted in the same migratory behaviour as control cells using. (B) Oncogenically transformed breast cells (MCF10a with H-Ras) with HSF1 are significantly more migratory than those without HSF1 (WT: ***P<0.001, ΔRDT: **P<001). Greater migratory capacity is a hallmark of metastatic cancer.

FIG. 7: Overexpression of HSF1 Results in More Disorganised Growth of Oncogenically Transformed Breast Cells in 3D Growth Conditions

(A) No significant change in 3D growth was observed in ‘normal’ MCF10a breast cells (mCherry) when the level of HSF1 (HSF1 WT and ΔRDT) was increased. In oncogenically transformed breast cells (H-RasV12) increasing stress through upregulated HSF1 (HSF1 WT and ΔRDT) resulted in significantly more disorganised growth in 3D culture which is characteristic of more metastatic cancer types. (B) The cell growth viewed at higher magnification under bright field (BF) lense, and with stains for Dapi/-catenin and Dapi-LamininV with shows the same results observed in A.

FIG. 8: Analysis of EGR4 Isoforms

Schematic diagram of the Human EGR4 gene, located on Chromosome 2, showing its 2 exon structure, the 3 possible mRNA transcripts and 3 hypothetical protein isoforms arising from these different transcripts. (B) Biostructural analysis of the 3 protein isoforms of EGR4 reveals 2 potential proteins (EGR4-1 and EGR4-S) and one unlikely possibility (EGR4-2) (C) Schematic diagram of the longer EGR4-1 isoform showing two proline-rich regions and multiple phosphorylation sites (D) Schematic diagram of the shorter/truncated EGR4-S isoform with one single proline-rich region and less phosphorylation sites.

FIG. 9: Confirmation of EGR4-S Splice Variants

(A) Schematic diagram of the 3 potential EGR4 mRNA transcripts. Two sets of primers were designed for different areas of the EGR4 mRNA. qPCR Amplicon 1 (A1) spans the intron/exon boundary whereas PCR Amplicon 2 (A2) sits entirely within exon 2. (B) Table containing details of the primers designed to produce amplicons 1 and 2, along with their binding sites on the full-length mRNA sequence (C) qPCR amplification to detect EGR4 and EGR4-S in in breast tumours. Results show the amplification cycle for qPCR product (mean±SEM) detected using primers binding in Amplicon 1 (“EXON 1”) vs primers binding in Amplicon 2/“EXON 2” for the same tumour sample. Amplicon 2/EXON 2 cycle products were detected at a cycle threshold indicating abundant EGR4-S nucleic acid in the samples, and Amplicon 1/EXON 1 cycle products were detected at a cycle threshold indicating no EGR4 nucleic acid in the samples.

FIG. 10: EGR4-S Expression is Localised to the Nucleus and Correlates with HER2 Overexpression

(A) Representative punch biopsies from a patient with HER2+ breast cancer stained for protein expression shows nuclear localisation of EGR4-S and membranous localisation of HER2 in the tumour cells. (B) HSF1 protein expression is also localised to the nucleus of tumour cells, consistent with it being a transcription factor. An example H&E stain from the samples is also presented. (C) A schematic of the EGFR(HER1)/HER2 signalling pathway and its relationship with the downstream transcription factor EGR4. Activation of the EGFR/HER2 pathway is linked to EGR4-S expression is also correlated with cancer cell growth. Downregulation of EGR4-S expression (eg via molecular stress) is correlated with drug resistance and metastasis. Representative punch biopsies from a patient with HER2−breast cancer exhibit no expression of HER2 protein and no nuclear expression of EGR4 (Data not shown).

FIG. 11: EGR4-S is Expressed in Tumour Tissue but not Adjacent Normal Tissue from the Same Patient

Example Western blot results for HER2 and EGR4 expression in normal (N) and breast tumour (T) biopsies from HER2− (pt 1-4) and HER2+(pt 5-9) patients. The EGR4-S variant was only detected in the HER2+ tumour tissue and not from patient-matched normal tissue or in any of the normal tissue examined. The higher molecular weight form of EGR4 is more visible across the normal tissue samples.

FIG. 12: EGR4-S Expression is Increased in Drug-Resistant Cells.

Shown is protein expression for HER2+ breast cancer cells (SKBR3) grown in increasing concentrations of Lapatinib over the course of 7 weeks. Week 1 shows the level of EGR4-S after growing the cells for 1 week but prior to any drug treatment (0 nM Lapatinib). Week 5 shows the cells after an additional 2 weeks treatment with a higher concentration of Lapatinib (300 nM) and these cells express less EGR4-S compared to the control. Note that as the cells tolerate increasing concentrations of the drug, the EGR4-S expression is greater. Week 7 shows the cells growing for an extra 2 weeks in even higher levels of Lapatinib (600 nM). These cells are growing in very high concentrations of Lapatinib and EGR4-S expression is returning to control levels.

FIG. 13: RNA-Seq Data Indicates EGR4-S is Expressed in Basal, HER2, Luminal A and Luminal B Breast Cancer Subtypes.

The dataset used in this study was extracted from the publicly available TCGA data set of mammary adenocarcinoma downloaded from Genomic data commons legacy archive (https://portal.gdc.cancer.gov/legacy-archive). Available clinical information from n=1085 patients was obtained for analysis. Samples were obtained from patients with initial diagnosis of invasive breast adenocarcinoma undergoing surgical resection and that had no prior treatment for their diseases. Samples were collected between 1988 and 2013, disregarding gender, race, histological type, disease stage or other co-morbidities. This figure shows the distribution and median proportional change for expression of EGR4 exon 1 and exon 2 m RNA relative to normal tissue. Expression of mRNA is separated according to breast cancer sub-type. Note that Exon 1 (present only in EGR4, not EGR4-S) was not detected in many samples analysed.

FIG. 14: RNA-Seq Analysis of EGR4 Exon Expression

Shown is RNAseq analysis for EGR4 exon expression in 56 different breast cancer cell lines. Reads were normalised to exon size by scaling to base level coverage. The majority of the cell lines included in the dataset had detectable levels of EGR4 mRNA (n=41 of 56). Of the 56 cell lines, 28 were found to only express exon 2, 13 to express both exons 1 and 2, two cell lines expressed exon 1 alone, and 13 had no detectable expression of EGR4. In cell lines where both exons were detected, the expression of exon 2 was much higher than exon 1 in all cases.

FIG. 15: EGR4-S Expression is Responsive to HER-Targeted Treatment and Affected by HSF1.

(A) Lapatinib treatment of HER2+ cells with/without elevated HSF1 and (B) with/without lowered HSF1 (C) (D) Comparison of HER2 and EGR4-S protein expression in tumour [T] tissue and adjacent normal [N] tissue from 8 different breast cancer patients (Pt 1-8) diagnosed with HER2+ tumours. Pt 11, 15 and 17 received pre-biopsy treatment (+PBT) to suppress the HER2 pathway.

### SUMMARY OF THE INVENTION

In one aspect, the present invention provides a method of predicting the responsiveness of a subject with cancer to treatment with a modulator of the HER signalling pathway, wherein the method comprises detecting the level of expression of EGR4-S in a sample of the subject, wherein the subject is likely to be responsive to treatment with the modulator of the HER signalling pathway if EGR4-S expression is at or above a predetermined level in the sample of the subject, and wherein the subject is likely to be resistant to treatment with the modulator of the HER signalling pathway if EGR4-S expression is at or below a predetermined level in the sample of the subject.

In another aspect, the present invention provides a method for predicting the responsiveness of a HER expressing tumour to treatment with a modulator of the HER signalling pathway, wherein the method comprises detecting the level of expression of EGR4-S in a sample of said tumour of a subject with cancer, wherein the tumour is likely to be responsive to treatment with the modulator of the HER signalling pathway if EGR4-S expression is at or above a predetermined level in the tumour sample, and wherein the tumour is likely to be resistant to treatment with the modulator of the HER signalling pathway if EGR4-S expression is at or below a predetermined level in the tumour sample.

In a further aspect, the present invention provides a method of identifying a subject with cancer who is likely to be responsive to treatment with a modulator of the HER signalling pathway, wherein the method comprises;
a) providing a sample of the subject;
b) detecting the level of expression of EGR4-S in the sample of the subject, and
c) identifying the subject as being likely to be responsive to treatment with the modulator of the HER signalling pathway if EGR4-S expression is at or above a predetermined level in the sample of the subject, and wherein the subject is likely to be resistant to treatment with the modulator of the HER signalling pathway if EGR4-S expression is at or below a predetermined level in the sample of the subject.

In one embodiment the present invention provides a method as described herein, further comprising administering to a subject identified as being likely to be responsive to treatment with the modulator of the HER signalling pathway a therapeutically effective amount of the modulator of the HER signalling pathway.

In another aspect, the present invention provides a method of treating or preventing a HER pathway activated cancer; wherein the method comprises;
a) providing a sample of the subject;
b) detecting the level of expression of EGR4-S in the sample of the subject;
c) identifying the subject as being likely to be responsive to treatment with the modulator of the HER signalling pathway if EGR4-S expression is at or above a predetermined level in the sample of the subject; and
d) administering to a subject identified as being likely to be responsive to treatment with the modulator of the HER signalling pathway a therapeutically effective amount of the modulator of the HER signalling pathway.

In one embodiment the present invention provides a method as described herein, further comprising detecting the level of expression of HSF1 in the sample of the subject.

In one embodiment the present invention provides a method as described herein, wherein the subject is likely to be resistant to treatment with the modulator of the HER signalling pathway if EGR4-S expression is at or below a predetermined level in the sample of the subject, and HSF1 expression is at or above a predetermined level in the sample of the subject.

In one embodiment the present invention provides a method as described herein, wherein the subject is likely to be responsive to treatment with the modulator of the HER signalling pathway if EGR4-S expression is at or above a predetermined level in the sample of the subject, and HSF1 expression is at or below a predetermined level in the sample of the subject.

In one embodiment the present invention provides a method as described herein, wherein the method further comprises detecting the level of expression of HER1 and/or HER2 in a sample of the subject.

In one embodiment the present invention provides a method as described herein, wherein the subject is likely to be responsive to treatment with the modulator of the HER signalling pathway if EGR4-S, and HER1 and/or HER2, is at or above a predetermined level in the sample of the subject.

In one embodiment the present invention provides a method as described herein, wherein the predetermined level is the amount or level of EGR4-S, HSF1, HER1 and/or HER2 in a control or reference sample.

In one embodiment the present invention provides a method as described herein, wherein the control or reference sample is a sample of cells obtained from: a normal healthy individual or individuals; a patient or patients with an analogous cancer; normal tissue adjacent to tumour tissue of the same subject; tumour cells of the same subject provided at a different time; or a cell line.

In one embodiment the present invention provides a method as described herein, wherein the modulator of the HER signalling pathway is an EGFR inhibitor.

In one embodiment the present invention provides a method as described herein, wherein the step of detecting the level of expression of EGR4-S in the sample comprises using PCR or RT-PCR.

In one embodiment the present invention provides a method as described herein, wherein the step of detecting the level of expression of EGR4-S in the sample comprises using PCR or RT-PCR across the EGR4 exon 1/exon 2 splice site.

In one embodiment the present invention provides a method as described herein, wherein the step of detecting the level of expression of EGR4-S in the sample comprises using FISH.

In one embodiment the present invention provides a method as described herein, wherein the step of detecting the level of expression of EGR4-S in the sample comprises using an immunohistochemical assay for EGR4-S polypeptide expression.

In one embodiment the present invention provides a method as described herein, wherein the step of detecting the level of expression of EGR4-S in the sample comprises using histology, ELISA, an ELISA-like assay, Western Blot, and/or flow cytometry to assay for EGR4-S polypeptide expression.

In one embodiment the present invention provides a method as described herein, wherein the cancer is a solid tumour.

In one embodiment the present invention provides a method as described herein, wherein the solid tumour is breast cancer tumour or lung cancer.

In one embodiment the present invention provides a method as described herein, wherein the method is performed prior to, concurrent with and/or following treatment with a modulator of the HER signalling pathway.

In one embodiment the present invention provides a method as described herein, wherein the modulator of the HER signalling pathway is administered by at least one route selected from orally, intravenously, intramuscularly, subcutaneously, topically or a combination thereof.

In one embodiment the present invention provides a method as described herein, wherein the subject likely to be resistant to treatment with the modulator of the HER signalling pathway has metastatic cancer.

In one embodiment the present invention provides a method as described herein, further comprising administering to a subject identified as being likely to be resistant to treatment with the modulator of the HER signalling pathway a therapeutically effective amount of the a non-HER signalling pathway anti-cancer therapy.

In a further aspect, the present invention provides a method of treating or preventing a HER pathway activated cancer; wherein the method comprises administering to a subject a therapeutically effective amount of a modulator of EGR4-S activity.

In a further aspect, the present invention provides a method of treating or preventing growth of a tumour of a HER pathway activated cancer; wherein the method comprises administering to a subject a therapeutically effective amount of a modulator of EGR4-S activity.

In one embodiment the present invention provides a method as described herein, wherein the modulator of EGR4-S activity is selected from the group consisting of an EGFR inhibitor, an siRNA, and a shRNA.

### DETAILED DESCRIPTION

The present invention is based in part on the characterisation splice variant of a stem cell transcription factor, Early Growth Response 4 (EGR4), designated EGR4-S, that is regulated by molecular stress. The present inventors have demonstrated that EGR4-S is upregulated strongly in HER pathway-activated breast cancer cell lines and that EGR4-S in these cells is responsive to HER pathway-targeted treatments (e.g. TKI drugs, HSP90 inhibitors) as well as modulators of stress (e.g. stress-inducing treatments such as HSP modulators, HSP inhibitors and Sulfurophane).

In particular, the present inventors have characterised a novel splice variant of a stem cell transcription factor (EGR4-S) that is not previously associated with cancer. Interestingly, the protein detected was not the predicted full-length form of EGR4 (expected to be more than 60 kDa) but rather a shortened version (51 kDa) that is responsive to cell stress.

For example, Example 3 demonstrates EGR4-S is regulated by HSF1 in normal and oncogenically transformed cells, and EGR4-S expression decreases with increasing HSF1 expression in oncogenically transformed breast cells. Example 4 demonstrates EGR4-S expression is increased in breast cancer cell lines, EGR4-S expression is increased in HER2+ breast cancer, and EGR4-S expression is decreased in HSF1 expressing breast cancer. Example 5 demonstrates EGR4-S is correlated with HER2+ and Basal breast cancer relapse-free survival. Example 6 demonstrates that EGR4-S expression is responsive to treatment with modulators of the HER signalling pathway in HER2+ cells.

Furthermore, HER-activated breast cancer cells with elevated levels of stress exhibited lower EGR4-S, lower growth potential but higher metastatic potential. Knocking down expression of EGR4-S in HER-activated cancer cells reduced proliferation of these cells. The responsiveness of EGR4-S to drug treatments is an indicator of drug sensitivity in these cancer cells. This work demonstrates that, when EGR4-S is down-regulated by stress, the cells become more metastatic and treatment is not as effective. Taken together, these results show that EGR4-S can be an excellent biomarker for cancer therapy, especially HER pathway-targeted treatments. Without wishing to be bound by theory, the present inventors propose that EGR4 is an indicator of stress-enhanced metastasis within the cancer cell and is a potential therapeutic target for cancer treatment in general.

For example, FIG. 3 demonstrates that expression of the transcription factor isoform EGR4-S is regulated by lapatinib, gefitinib and erlotinib, modulators of the HER signalling pathway. This data indicates that cancer cells expressing the transcription factor isoform EGR4-S respond to modulators of the HER signalling pathway, indicating EGR4-S expression can be used as a marker of responsiveness. Importantly the present inventors have demonstrated in FIG. 5 that decreasing EGR4-S expression using shRNA decreases cancer cell growth. Example 5 demonstrates HSF1 expression is a marker of increased molecular stress in cancer cells and EGR4-S expression is a marker of decreased molecular stress in cancer cells, indicating that HSF1 and/or EGR4-S expression can be used as a marker of molecular stress in cancer cells and/or metastasis.

Furthermore, FIG. 4 demonstrates that increased HSF1 expression is associated with hallmarks of metastatic cancer.

The term “metastasis” as used herein refers to the process by which a disease shifts from one part of the body to another. This process may include the spreading of neoplasms from the site of a primary tumour to distant parts of the body. The term “metastatic cancer” refers to any cancer in any part of the body which has its origins in primary cancer at a site distant from the location of the secondary tumour. Metastatic cancer includes, but is not limited to true “metastatic tumours” as well as pre-metastatic primary tumour cells in the process of developing a metastatic phenotype.

Accordingly, in one embodiment the present invention provides a method of predicting the responsiveness of a subject with cancer to treatment with a modulator of the HER signalling pathway, wherein the method comprises detecting the level of expression of EGR4-S in a sample of the subject, wherein the subject is likely to be responsive to treatment with the modulator of the HER signalling pathway if EGR4-S expression is at or above a predetermined level in the sample of the subject, and wherein the subject is likely to be resistant to treatment with the modulator of the HER signalling pathway if EGR4-S expression is at or below a predetermined level in the sample of the subject.

As used herein, the term “predicting” includes to determine or tell in advance. When used to “predict” the responsiveness to a treatment, for example, treatment with a modulator of the HER signalling pathway, anti-cancer treatment etc, the term “predict” can mean that the likelihood of the outcome of the treatment can be determined at the outset, before the treatment has begun, before the treatment period has progressed substantially, or during the course of treatment with one or more cancer treatments (e.g. monitoring). A predictive method may also be referred to herein as a prognostic method.

As used herein, the term “responsiveness” or “responsive,” when used in reference to a treatment includes, for example, treatment with a modulator of the HER signalling pathway, anti-cancer treatment etc, refers to the degree of effectiveness of the treatment in lessening or decreasing the symptoms of a disease, disorder, or condition being treated. For example, the term “increased responsiveness,” when used in reference to a treatment of a subject, a tumour, or a cell, refers to an increase in the effectiveness in lessening or decreasing the symptoms of the disease when measured using any methods known in the art. In some embodiments, the increase in the effectiveness is at least about 5%, at least about 10%, at least about 20%, at least about 30%, at least about 40%, or at least about 50%.

In one embodiment the present invention provides a method as described herein wherein a subject with cancer is selected for an anti-cancer treatment, wherein the patient is selected based on the level of expression of EGR4-S in a sample of a tumour of the subject with cancer.

In one embodiment the present invention provides a method as described herein wherein a subject with cancer is selected for an anti-cancer treatment, wherein the patient is selected based on the level of expression of EGR4-S, HSF1, HER1 and/or HER2 in a sample of a tumour of the subject with cancer.

As used herein, an “anti-cancer treatment” includes a drug used to treat cancer. Non-limiting examples of anti-tumor agents herein include chemotherapeutic agents, HER inhibitors, HER dimerization inhibitors, HER antibodies, antibodies directed against tumor associated antigens, anti-hormonal compounds, cytokines, EGFR-targeted drugs, anti-angiogenic agents, tyrosine kinase inhibitors, growth inhibitory agents and antibodies, cytotoxic agents, antibodies that induce apoptosis, COX inhibitors, farnesyl transferase inhibitors, antibodies that binds oncofetal protein CA 125, HER2 vaccines, Raf or ras inhibitors, liposomal doxorubicin, topotecan, taxane, dual tyrosine kinase inhibitors, TLK286, EMD-7200, pertuzumab, trastuzumab, erlotinib, and bevacizumab.

For example, a subject, cancer type and/or tumour which is able to respond (“responsive”) to a modulator of the HER signalling pathway is one which when treated with a modulator (e.g. a HER inhibitor), such as a HER2 antibody or small molecule inhibitor, shows a therapeutically effective benefit in the subject according to any of the criteria for therapeutic effectiveness known to the skilled oncologist, including those elaborated herein, but particularly in terms of survival, including progression free survival (PFS) and/or overall survival (OS).

As used herein the term response is used interchangeably with benefit or clinical benefit or efficacy can be measured by any method known in the art. For example, the response to a therapy, relates to any response of the cancer, e.g., a tumour, to the therapy, preferably to a change in tumour mass and/or volume after initiation of treatment, e.g. neoadjuvant or adjuvant chemotherapy. Tumour response may be assessed in a neoadjuvant or adjuvant situation where the size of a tumour after systemic intervention can be compared to the initial size and dimensions as measured by CT, PET, mammogram, ultrasound or palpation and the cellularity of a tumour can be estimated histologically and compared to the cellularity of a tumour biopsy taken before initiation of treatment. A response may also be assessed by caliper measurement or pathological examination of the tumour after biopsy or surgical resection. A response may be recorded in a quantitative fashion like percentage change in tumour volume or cellularity or using a semi-quantitative scoring system such as residual cancer burden. Assessment of tumour response may be performed early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumour cells and/or the tumour bed.

In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy.

Additional criteria for evaluating the response to a modulator of the HER signalling pathway are related to “survival,” which includes all of the following: survival until mortality, also known as overall survival (wherein said mortality may be either irrespective of cause or tumour related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumour recurrence.

As used herein the term “modulator of the HER signalling pathway” includes an agent that modulates an activity of a HER signalling pathway. The term includes small molecules, antibodies, antigen binding fragments thereof, immunoadhesins, fusion proteins, oligopeptides and other molecules that decrease, block, inhibit, abrogate or interfere with signal transduction resulting from the interaction of the molecules of the HER family, including siRNA, shRNA and other interfering RNA.

Human epidermal growth factor receptor (HER) signalling has been linked to cancer. As used herein, the term “HER-signalling pathway” or “HER-pathway” refers to one or more HER pathways. The HER family consists of 4 structurally related cellular receptors, which interact in many ways. They include HER1 (EGFR), HER2, HER3, and HER4. After ligand binding, receptors dimerize, or form pairs. Upon dimerization, the intracellular tyrosine kinase domains of the receptors are phosphorylated, activating the receptors and initiating downstream signalling cascades, such as the MAPK proliferation pathway and/or the PI3K/Akt prosurvival pathway. With 4 members of the HER family of receptors, each of which is able to homodimerize or to heterodimerize with other members of the HER family, multiple receptor combinations are possible. HER2 is the preferred dimerization partner for all members of the HER receptor family since it exists in an open conformation and is continually available for dimerization. Accordingly, an activity of a HER signalling pathway includes an activity of HER1 (EGFR), HER2, HER3, and HER4, and an activity of a downstream signalling cascade, including the MAPK proliferation pathway and/or the PI3K/Akt prosurvival pathway.

EGR4 protein analysis using UniProt (uniprot.org) shows that MET-1 or MET-104 could be the first initiation site of the EGR4 protein product. EGR4 is a transcriptional regulator, and key areas of the protein for DNA binding are 3 zinc finger regions (located at positions 483-507, 513-535 and 541-563) all located in exon 2. The protein sequence of the full length EGR4 below shows zinc finger DNA binding regions highlighted:

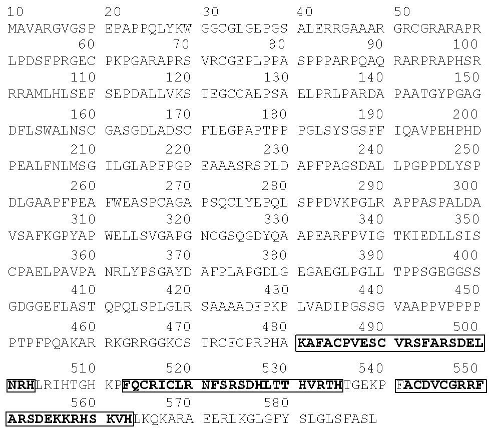

Without wishing to be bound by theory, EGR4 preferentially binds to an EGR consensus motif (5′-GCGG/TGGGCG-3′), regulates brain-derived neurotrophic factor (BDNF)-mediated neuron-specific potassium chloride cotransporter 2 (KCC2) transcription via the ERK1/2 signalling pathway in immature neurons, binds to nuclear factor activated T cells (NFAT) or nuclear factor kappa B (NFκB) to enhance the transcription of downstream genes encoding inflammatory cytokines, such as IL-2, TNF-α and ICAM-1, and directly regulates the transcriptional activity of the PTHrP gene. Furthermore, a number of downstream genes have been identified by microarray analysis. For example:

| | | | | FC | | FC | |
|---|---|---|---|---|---|---|---|
| Probe ID | Accession no. | Symbol | Gene Name | 48 h | P-value | 72 h | P-value |
| A_23_P108948 | NM_018000 | MREG | melanoregulin | −3.12 | 5.62E−03 | −2.51 | 1.40E−03 |
| A_23_P21363 | NM_024060 | AHNAK | AHNAK | −2.70 | 4.64E−03 | −3.04 | 1.33E−03 |
| | | | nucleoprotein | | | | |
| A_24_P602871 | NM_001030060 | SAMD5 | sterile alpha motif | −2.50 | 1.37E−02 | −2.15 | 6.47E−03 |
| | | | domain containing 5 | | | | |
| A_24_P193295 | NM_198686 | RAB15 | RAB15, member RAS | −2.44 | 1.93E−03 | −2.01 | 1.04E−02 |
| | | | onocogene family | | | | |
| A_24_P328675 | NM_015466 | PTPN23 | protein tyrosine | −2.36 | 2.02E−03 | −2.17 | 1.58E−03 |
| | | | phosphatase, non- | | | | |
| | | | receptor type 23 | | | | |
| A_23_P86195 | NM_152369 | SLC44A3 | solute carrier | −2.34 | 3.66E−03 | −2.01 | 9.50E−03 |
| | | | family 44, member 3 | | | | |
| A_23_P77103 | NM_003104 | SORD | sorbitol dehydrogenase | −2.34 | 3.68E−03 | −2.22 | 3.59E−03 |
| A_23_P344531 | NM_007286 | SYNPO | synaptopodin | −2.32 | 2.56E−03 | −2.01 | 2.01E−03 |
| A_32_P89691 | NM_003104 | SORD | sorbitol dehydrogenase | −2.30 | 4.97E−03 | −2.19 | 4.09E−04 |
| A_32_P127153 | NM_003104 | SORD | sorbitol dehydrogenase | −2.27 | 1.02E−03 | −2.12 | 4.45E−03 |
| A_24_P179316 | NM_015466 | PTPN23 | protein tyrosine | −2.27 | 8.56E−03 | −2.01 | 1.20E−02 |
| | | | phosphatase, non- | | | | |
| | | | receptor type 23 | | | | |
| A_23_P386561 | NM_001002926 | TWISTNB | TWIST neighbor | −2.26 | 4.19E−03 | −2.32 | 8.88E−04 |
| A_23_P213518 | NM_001042440 | CAST | calpastatin | −2.25 | 3.41E−03 | −2.11 | 3.49E−03 |
| A_23_P212329 | NM_015466 | PTPN23 | protein tyrosine | −2.21 | 1.79E−03 | −2.24 | 2.76E−04 |
| | | | phosphatase, non- | | | | |
| | | | receptor type 23 | | | | |
| A_32_P187009 | NM_001174072 | SERINC5 | serine incorporator 5 | −2.18 | 2.41E−03 | −2.13 | 3.02E−03 |
| A_23_P82474 | NM_001002926 | TWISTNB | TWIST neighbor | −2.08 | 1.23E−02 | −2.08 | 1.96E−03 |
| A_23_P132595 | NM_014667 | VGLL4 | vestigial like 4 | −2.05 | 6.20E−03 | −2.02 | 1.27E−03 |
| | | | (Drosophila) | | | | |
| A_23_P318262 | NM_005221 | DLX5 | distal-less homeobox 5 | −2.05 | 2.03E−03 | −2.08 | 3.68E−03 |

P-value, Benjamini-Hochberg false discovery rate of random permutation test; fold change (FC), ratio of gene expression level between siEGFP and siEGR4; Gene symbol, accession number and gene name, exported from GeneSpring (from the NCBI databases).

Accordingly, an activity of a HER signalling pathway includes an activity of expression of and/or an activity of a downstream gene or protein encoded by a downstream gene described herein.

A modulator of the HER signalling pathway includes a molecule that decreases, blocks, inhibits, abrogates or interferes with signal transduction resulting from the interaction of a member of the HER family with one or more of its binding partners. In some embodiments, the modulator is an antagonist that inhibits the binding of a member of the HER family to its binding partner(s).

The term “detecting” as used herein include any form of measurement, and includes detecting the expression, including, for example, expression of an EGR4 exon, EGR4-S mRNA, EGR4-S, HSF1, HSF-1 mRNA, HER1, HER1 mRNA, HER2, HER2 mRNA etc. in the sample, a cell, a tumour cell, etc. as disclosed herein. The term “detecting” includes both quantitative and/or qualitative determination. Expression may be determined by any suitable method known to those skilled in the art, including those as further disclosed herein. As used herein the term “detecting” includes any means of detecting, including direct and indirect detection. For example, a molecule described herein can be detected using an antibody, for example an EGR4S, a HSF1, a HER1 and/or a HER2 antibody. The presence and/or expression level/amount of molecule in a sample can be analyzed by a number of methodologies, many of which are known in the art and understood by the skilled artisan, including, but not limited to, immunohistochemistry (“IHC”), Western blot analysis, immunoprecipitation, molecular binding assays, ELISA, ELIFA, fluorescence activated cell sorting (“FACS”), MassARRAY, proteomics, quantitative blood based assays (as for example Serum ELISA), biochemical enzymatic activity assays, in situ hybridization, Southern analysis, Northern analysis, whole genome sequencing, polymerase chain reaction (“PCR”) including quantitative real time PCR (“qRT-PCR”) and other amplification type detection methods, such as, for example, branched DNA, SISBA, TMA and the like, RNA-Seq, FISH, microarray analysis, gene expression profiling, and/or serial analysis of gene expression (“SAGE”), as well as any one of the wide variety of assays that can be performed by protein, gene, and/or tissue array analysis. Typical protocols for evaluating the status of genes and gene products are found, for example in Ausubel et al., eds., 1995, Current Protocols in Molecular Biology, Units 2 (Northern Blotting), 4 (Southern Blotting), 15 (Immunoblotting) and 18 (PCR Analysis). Multiplexed immunoassays such as those available from Rules Based Medicine or Meso Scale Discovery (“MSD”) may also be used.

“EGR4” was first characterised in the central nervous system (Crosby et al., 1991; Crosby et al., 1992), and research has since shown that this neural expression is transient in the vertebrate developing brain (Bae et al., 2015). Roberts and colleagues (2012) have shown that, within the brain, neurons can live for the entire lifespan of the host organism, and this lifespan extends when the neurons are transferred to a new host (Magrassie et al., 2013). Furthermore, EGR4 expression has also been isolated from human testicles where it is localised within stem cells that survive to produce spermatozoa across the lifespan (Hadziselimovic et al., 2009). Without wishing to be bound by theory, the present inventors propose that EGR4 in both these contexts is linked to cells that have longer lifespans than other body cells, and following the data described herein it is possible that EGR4-S expression is involved with cancer cells developing the characteristic of immortalisation. Just like stem cells, cancer cells, once established, have endless proliferative potential (Reya et al., 2001) enabled by the activation of pathways such as the HER pathway. The present inventors have demonstrated that EGR4-S is highly expressed in HER2+ breast cancer cell lines and that EGR4-S is a downstream effector of the HER signalling pathway.

EGR4, early growth response 4, is also known as AT133; NGFIC; NGFI-C; PAT133. The NCBI Gene ID for EGR4 is 1961, and the UniProtKB/Swiss-Prot ID for EGR4 is Q05215.

An exemplary EGR4 mRNA sequence is reproduced below:

GAGCCCGGCAGCGCTTTGGAGAGGCGAGGAGCCGCCGCCC
GAGGCCGGTGCGGGCGAGCGAGGGCGCCGCGGCTCCCCGA
CTCCTTTCCCAGAGGTGAGTGCCCGAAGCCAGGAGCCCGG
GCGCCTAGGTCTGTGCGCTGCGGGGAACCCCTACCGCCAG
CCTCCCCGCCACCCGCGCGCCCCCAAGCCCAGCGGGCGAG
GCCCCGGGCGCCCCACAGCCGGCGCCGCGCCATGCTCCAC
CTTAGCGAGTTTTCCGAACCCGACGCGCTCCTCGTCAAGT
CCACTGAAGGCTGTTGCGCCGAACCCAGCGCTGAATTGCC
CCGGCTGCCTGCCAGGGACGCTCCCGCGGCCACCGGCTAC
CCTGGAGCAGGCGACTTCTTGAGCTGGGCTTTGAACAGCT
GCGGCGCAAGTGGGGACTTAGCCGACTCCTGCTTCCTGGA
GGGGCCTGCGCCCACACCCCCTCCCGGCCTCAGCTACAGC
GGTAGCTTCTTCATTCAGGCAGTGCCCGAACACCCGCACG
ACCCGGAGGCACTCTTCAACCTCATGTCGGGCATCTTAGG
CCTGGCACCCTTCCCCGGTCCAGAGGCAGCAGCGTCCAGA
TCCCCGCTGGATGCCCCTTTTCCTGCGGGGTCCGATGCCT
TGCTGCCGGGTCCGCCGGACCTTTACTCCCCGGATCTGGG
CGCTGCCCCTTTCCCAGAGGCGTTCTGGGAGGCCTCGCCT
TGCGCGGGTGCCCCCTCGCAGTGCCTGTATGAGCCTCAGC
TCTCCCCGCCCGACGTCAAGCCCGGCCTCCGGGCGCCTCC
CGCCTCGCCAGCGCTGGACGCTGTCTCTGCCTTCAAGGGT
CCCTACGCGCCCTGGGAGCTGCTTTCTGTGGGGGCCCCAG
GGAACTGTGGGTCACAGGGAGACTACCAGGCCGCCCCGGA
GGCTCGTTTTCCCGTAATAGGGACCAAGATTGAGGACTTG
CTGTCCATCAGCTGCCCTGCGGAACTGCCGGCCGTCCCAG
CCAACAGACTCTATCCCAGCGGGGCCTATGACGCTTTCCC
GCTGGCCCCGGGTGACTTAGGGGAGGGGGCTGAGGGCCTC
CCTGGGCTCCTGACCCCTCCTAGTGGGGAGGGAGGGAGTA
GCGGCGACGGCGGAGAGTTTCTGGCCAGTACGCAGCCTCA
GCTTTCCCCGCTGGGCCTTCGCAGCGCCGCCGCGGCGGAC
TTCCCTAAACCTCTGGTGGCGGACATCCCTGGAAGCAGTG
GCGTGGCTGCACCACCCGTGCCGCCGCCGCCGCCCACCCC
TTTCCCCCAGGCCAAGGCGCGACGCAAGGGGCGCCGCGGC
GGCAAATGCAGCACGCGCTGCTTCTGCCCGCGGCCGCACG
CCAAGGCCTTCGCTTGCCCGGTGGAGAGTTGTGTGCGGAG
CTTTGCGCGCTCCGACGAGCTCAATCGCCACCTGCGCATC
CACACGGGCCACAAACCCTTCCAGTGCCGCATCTGCCTCC
GCAACTTCAGCCGCAGCGACCACCTCACCACGCACGTGCG
CACCCACACCGGCGAGAAGCCTTTTGCTTGCGACGTGTGC
GGCCGCCGCTTCGCGCGCAGCGATGAGAAGAAACGGCACA
GCAAGGTGCACCTCAAGCAGAAGGCGCGCGCCGAGGAGCG
GCTCAAGGGCCTCGGCTTTTACTCGCTGGGCCTCTCCTTC
GCTTCTCTCTGAGCAAGAGATGGGTTTATGGGTTGGGGCG
CCGCCGTTCGGCGCGCACGAGTTCCGGGCCGTTCCCCTCC
CCGCTCTTCTTCCAACTCCTCCTCGCACGCCCGAGGGCCG
GCCTCCGGTCCCGCTTCCAGTTTCCTTGAAGCGCCCGCCG
CACACGCCCTATTCAGCACCAGCTCCGCGGACAGTTCCCG
CGGTCCAGGCGCTGTCACCCTTGTCAGCCGCGCTTTGGGG
GAAGTCTTCTGAGACCACCCAGTGAATAGGCACTACCCTG
GGATTCAAGACAGTCTTTTGTAACTGGCACACGCCCCACG
CCTTCCTCTATAACCCCCAGAGACAGGCTGGGGCAGCGCC
AAGGCGGTCTCGCGCGGGACTTTGTACAGCAGTGTCTTAT
CCAGCAGCCATTGGATGTAACGTTTTGCTTTGGGTTTTTT
TTCCTTTTGTTGTTGTTAATTTTTGTAAAGCAGACGCTAC
TCTCAAGCAGTTGACAAAACTGTTTATTTTTGCAATTAAA
ATTATTGTGCTAAAAGCTTA

An exemplary EGR4 amino acid sequence is reproduced below:

MLHLSEFSEPDALLVKSTEGCCAEPSAELPRLPARDAPAA
TGYPGAGDFLSWALNSCGASGDLADSCFLEGPAPTPPPGL
SYSGSFFIQAVPEHPHDPEALFNLMSGILGLAPFPGPEAA
ASRSPLDAPFPAGSDALLPGPPDLYSPDLGAAPFPEAFWE
ASPCAGAPSQCLYEPQLSPPDVKPGLRAPPASPALDAVSA
FKGPYAPWELLSVGAPGNCGSQGDYQAAPEARFPVIGTKI
EDLLSISCPAELPAVPANRLYPSGAYDAFPLAPGDLGEGA
EGLPGLLTPPSGEGGSSGDGGEFLASTQPQLSPLGLRSAA
AADFPKPLVADIPGSSGVAAPPVPPPPPTPFPQAKARRKG
RRGGKCSTRCFCPRPHAKAFACPVESCVRSFARSDELNRH
LRIHTGHKPFQCRICLRNFSRSDHLTTHVRTHTGEKPFAC
DVCGRRFARSDEKKRHSKVHLKQKARAEERLKGLGFYSLG
LSFASL

As used herein, “EGR4S” refers to an isoform of EGR4 comprising only part of exon 2 of EGR4, as is shown in FIGS. 8 and 9. The present inventors propose that the EGR4-S amino acid sequence includes an amino acid sequence encoded by the region beginning with the start codon within exon 2.

An exemplary EGR4 mRNA sequence is reproduced below with the EGR4 start codon in bold italics and the predicted EGR4-S start codon in underlined bold italics:

GAGCCCGGCAGCGCTTTGGAGAGGCGAGGAGCCGCCGCCC
GAGGCCGGTGCGGGCGAGCGAGGGCGCCGCGGCTCCCCGA
CTCCTTTCCCAGAGGTGAGTGCCCGAAGCCAGGAGCCCGG
GCGCCTAGGTCTGTGCGCTGCGGGGAACCCCTACCGCCAG
CCTCCCCGCCACCCGCGCGCCCCCAAGCCCAGCGGGCGAG
GCCCCGGGCGCCCCACAGCCGGCGCCGCGCCATGCTCCAC
CTTAGCGAGTTTTCCGAACCCGACGCGCTCCTCGTCAAGT
CCACTGAAGGCTGTTGCGCCGAACCCAGCGCTGAATTGCC
CCGGCTGCCTGCCAGGGACGCTCCCGCGGCCACCGGCTAC
CCTGGAGCAGGCGACTTCTTGAGCTGGGCTTTGAACAGCT
GCGGCGCAAGTGGGGACTTAGCCGACTCCTGCTTCCTGGA
GGGGCCTGCGCCCACACCCCCTCCCGGCCTCAGCTACAGC
GGTAGCTTCTTCATTCAGGCAGTGCCCGAACACCCGCACG
ACCCGGAGGCACTCTTCAACCTCATGTCGGGCATCTTAGG
CCTGGCACCCTTCCCCGGTCCAGAGGCAGCAGCGTCCAGA
TCCCCGCTGGATGCCCCTTTTCCTGCGGGGTCCGATGCCT
TGCTGCCGGGTCCGCCGGACCTTTACTCCCCGGATCTGGG
CGCTGCCCCTTTCCCAGAGGCGTTCTGGGAGGCCTCGCCT
TGCGCGGGTGCCCCCTCGCAGTGCCTGTATGAGCCTCAGC
TCTCCCCGCCCGACGTCAAGCCCGGCCTCCGGGCGCCTCC
CGCCTCGCCAGCGCTGGACGCTGTCTCTGCCTTCAAGGGT
CCCTACGCGCCCTGGGAGCTGCTTTCTGTGGGGGCCCCAG
GGAACTGTGGGTCACAGGGAGACTACCAGGCCGCCCCGGA
GGCTCGTTTTCCCGTAATAGGGACCAAGATTGAGGACTTG
CTGTCCATCAGCTGCCCTGCGGAACTGCCGGCCGTCCCAG
CCAACAGACTCTATCCCAGCGGGGCCTATGACGCTTTCCC
GCTGGCCCCGGGTGACTTAGGGGAGGGGGCTGAGGGCCTC
CCTGGGCTCCTGACCCCTCCTAGTGGGGAGGGAGGGAGTA
GCGGCGACGGCGGAGAGTTTCTGGCCAGTACGCAGCCTCA
GCTTTCCCCGCTGGGCCTTCGCAGCGCCGCCGCGGCGGAC
TTCCCTAAACCTCTGGTGGCGGACATCCCTGGAAGCAGTG
GCGTGGCTGCACCACCCGTGCCGCCGCCGCCGCCCACCCC
TTTCCCCCAGGCCAAGGCGCGACGCAAGGGGCGCCGCGGC
GGCAAATGCAGCACGCGCTGCTTCTGCCCGCGGCCGCACG
CCAAGGCCTTCGCTTGCCCGGTGGAGAGTTGTGTGCGGAG
CTTTGCGCGCTCCGACGAGCTCAATCGCCACCTGCGCATC
CACACGGGCCACAAACCCTTCCAGTGCCGCATCTGCCTCC
GCAACTTCAGCCGCAGCGACCACCTCACCACGCACGTGCG
CACCCACACCGGCGAGAAGCCTTTTGCTTGCGACGTGTGC
GGCCGCCGCTTCGCGCGCAGCGATGAGAAGAAACGGCACA
GCAAGGTGCACCTCAAGCAGAAGGCGCGCGCCGAGGAGCG
GCTCAAGGGCCTCGGCTTTTACTCGCTGGGCCTCTCCTTC
GCTTCTCTCTGAGCAAGAGATGGGTTTATGGGTTGGGGCG
CCGCCGTTCGGCGCGCACGAGTTCCGGGCCGTTCCCCTCC
CCGCTCTTCTTCCAACTCCTCCTCGCACGCCCGAGGGCCG
GCCTCCGGTCCCGCTTCCAGTTTCCTTGAAGCGCCCGCCG
CACACGCCCTATTCAGCACCAGCTCCGCGGACAGTTCCCG
CGGTCCAGGCGCTGTCACCCTTGTCAGCCGCGCTTTGGGG
GAAGTCTTCTGAGACCACCCAGTGAATAGGCACTACCCTG
GGATTCAAGACAGTCTTTTGTAACTGGCACACGCCCCACG
CCTTCCTCTATAACCCCCAGAGACAGGCTGGGGCAGCGCC
AAGGCGGTCTCGCGCGGGACTTTGTACAGCAGTGTCTTAT
CCAGCAGCCATTGGATGTAACGTTTTGCTTTGGGTTTTTT
TTCCTTTTGTTGTTGTTAATTTTTGTAAAGCAGACGCTAC
TCTCAAGCAGTTGACAAAACTGTTTATTTTTGCAATTAAA
ATTATTGTGCTAAAAGCTTA

An exemplary predicted EGR4-S amino acid sequence is reproduced below:

MSGILGLAPFPGPEAAASRSPLDAPFPAGSDALLPGPPDL
YSPDLGAAPFPEAFWEASPCAGAPSQCLYEPQLSPPDVKP
GLRAPPASPALDAVSAFKGPYAPWELLSVGAPGNCGSQGD
YQAAPEARFPVIGTKIEDLLSISCPAELPAVPANRLYPSG
AYDAFPLAPGDLGEGAEGLPGLLTPPSGEGGSSGDGGEFL
ASTQPQLSPLGLRSAAAADFPKPLVADIPGSSGVAAPPVP
PPPPTPFPQAKARRKGRRGGKCSTRCFCPRPHAKAFACPV
ESCVRSFARSDELNRHLRIHTGHKPFQCRICLRNFSRSDH
LTTHVRTHTGEKPFACDVCGRRFARSDEKKRHSKVHLKQK
ARAEERLKGLGFYSLGLSFASL

The present inventors have demonstrated in FIG. 9 that EGR4S can be detected using qPCR. Exemplary oligonucleotides for amplifying EGR4 across the intron/exon boundary are shown below:

EGR4-A1-Fwd
5′ CGTCAAGTCCACTGAAGGCT
EGR4-A1-Rev
5′ AAGCCCAGCTCAAGAAGTCG

These exemplary oligonucleotides do not amplify EGR4-S.

Exemplary oligonucleotides for amplifying exon 2 of EGR4 are shown below:

EGR4-A2-Fwd
5′ AGAGTTGTGTGCGGAGCTTT 3′
EGR4-A2-Rev
5′ CTGAAGTTGCGGAGGCAGAT 3′

These exemplary oligonucleotides amplify EGR4-S and EGR4.

The terms “level of expression” or “expression level” in general are used interchangeably and generally refer to the amount of a molecule (e.g. EGR4S or EGR4S RNA) in a biological sample. “Expression” generally refers to the process by which information (e.g., gene-encoded and/or epigenetic) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) ae also regarded as expressed. “Expressed genes” include those that are transcribed into a polynucleotide as mRNA and then translated into a polypeptide, and also those that are transcribed into RNA but not translated into a polypeptide.

“Increased expression,” “increased expression levels,” or “elevated levels” refers to an increased expression or increased levels of a molecule (e.g. EGR4S or EGR4S RNA etc) in an individual relative to a control, such as an individual or individuals who are not suffering from the disease or disorder (e.g., cancer) or an internal control (e.g., a control response or a control biomarker), or a predetermined level.

“Decreased expression,” “decreased expression levels,” or “reduced levels” refers to a decrease expression or decreased levels of a molecule (e.g. EGR4S or EGR4S RNA etc) in an individual relative to a control, such as an individual or individuals who are not suffering from the disease or disorder (e.g., cancer) or an internal control (e.g., a control response or a control biomarker) or a predetermined level. In some embodiments, decreased expression is little or no expression, as is discussed in further detail below.

As used herein the term “predetermined level”, or alternatively herein “threshold level” or “predetermined threshold level”) refers to a level of EGR4-S, HSF1, HER1, HER2, or any other molecule disclosed herein which may be of interest for comparative purposes. In some aspects, a threshold level may be the expression level of a protein or nucleic acid expressed as an average of the level of the expression level of a protein or nucleic acid from samples taken from a control population of healthy (disease-free) subjects.

In some aspects, the threshold level may be the level in the same subject at a different time, e.g. such as the level determined prior to the subject developing the disease or prior to initiating therapy. In general, samples are normalized by a common factor. For example, body fluid samples are normalized by volume body fluid and cell-containing samples are normalized by protein content or cell count. In another aspect, the threshold level may also refer to the level of expression of the same molecule in a corresponding control sample or control group of subjects which do not respond to treatment, or who do not have the disease.

In one embodiment the predetermined level is a cut-off or threshold against which the measured expression level of a protein or nucleic acid is compared. Based on comparison to known control samples, a “threshold level” for EGR4-S or HSF-1 or any other molecule disclosed herein can be determined, and test samples that fall above or below the molecule's threshold levels indicate that the patient from whom the sample was obtained may or may not benefit from treatment with a modulator of the HER signalling pathway.

For example, ERG4-S levels above its predetermined level in a sample would indicate that the patient may benefit from treatment with a modulator of the HER signalling pathway. In another example, ERG4-S levels below its predetermined level in a sample would indicate that the patient may not benefit from treatment with a modulator of the HER signalling pathway. In a further example, ERG4-S levels below its predetermined level in a sample would indicate that the patient may benefit from treatment with an anti-cancer therapy that is not a modulator of the HER signalling pathway. In another example, HSF1 levels below its predetermined level in a sample would indicate that the patient may benefit from treatment with a modulator of the HER signalling pathway. In another example, HSF1 levels above its predetermined level in a sample would indicate that the patient may not benefit from treatment with a modulator of the HER signalling pathway. In another example, HSF1 levels above its predetermined level in a sample would indicate that the patient may benefit from treatment with an anti-cancer therapy that is not a modulator of the HER signalling pathway.

In some aspects, predetermined threshold levels for any molecule (e.g. EGR4-S, HSF1 etc) can be predetermined and matched as to the type of sample (e.g. serum, tumour tissue), or type of cancer.

In other aspects, the predetermined threshold level is based on the median level in a sample measured from a plurality of patients having cancer.

As used herein, the terms “likelihood”, “likely to”, and similar includes an increase in the probability of an event. Accordingly, “likelihood”, “likely to”, and similar, when used in reference to responsiveness to cancer therapy, generally contemplates an increased probability that the individual will exhibit a reduction in the severity of cancer or a symptom of cancer or the or slowing of the cancer progression, e.g. cancer cell or tumour growth. The term “likelihood”, “likely to”, and similar, when used in reference to responsiveness to cancer therapy, can also generally mean the increase of indicators that may evidence an increase in the treatment of cancer.

As used herein the term “sample”, refers to a composition that is obtained or derived from a subject and/or individual of interest that contains a cellular and/or other molecular entity, or response that is to be characterized and/or identified, for example based on physical, biochemical, chemical and/or physiological characteristics, or response (including molecules such as EGR4S and HSF1 etc) described herein. For example, a sample or disease sample and variations thereof refers to any sample obtained from a subject of interest that would be expected or is known to contain the cellular and/or molecular entity, that is to be characterized. Samples include, but are not limited to cells derived from a subject, for example from whole blood, blood derived cells, or tumour or tissue derived cells, but are not limited to, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebrospinal fluid, saliva, sputum, tears, perspiration, mucus, tumour lysates, and tissue culture medium, tissue extracts such as homogenized tissue, tumour tissue, cellular extracts, cell-free DNA and/or cell-free RNA, or a combination thereof.

In one embodiment, the sample can comprise a tumour cell biopsy, or a plurality of samples from a clinical trial, etc.

The sample can be a crude sample, or can be purified to various degrees prior to storage, processing, or measurement.

In a preferred embodiment, the sample comprises a cell, a cell culture, a tissue, and/or a biological fluid, for example, tumour cells, whole blood, serum, plasma, a sample comprising cell-free DNA and/or cell-free RNA, or a combination thereof.

As used herein the terms “patient”, “subject” or “individual” includes any animal, or cells thereof whether in vitro or in situ, amenable to the methods described herein. Preferably, the patient is a human. In some embodiments, the subject is a subject in need of treatment thereof. In other embodiments, the subject has a HER-activated cancer. In some embodiments, the subject is a subject in need of treatment thereof. In other embodiments, the subject has a HER-pathway activated cancer.

In a preferred embodiment, the HER pathway activated cancer is a HER2+ cancer.

In another preferred embodiment, the HER pathway activated cancer is a HER1+ cancer.

In another preferred embodiment, the HER pathway activated cancer is a HER2+ EGR4S+ cancer.

In another preferred embodiment, the HER pathway activated cancer is a HER2+ EGR4S+ HSF1-low cancer.

In another embodiment, the present invention provides a method for predicting the responsiveness of a HER expressing tumour to treatment with a modulator of the HER signalling pathway, wherein the method comprises detecting the level of expression of EGR4-S in a sample of said tumour of a subject with cancer, wherein the tumour is likely to be responsive to treatment with the modulator of the HER signalling pathway if EGR4-S expression is at or above a predetermined level in the tumour sample, and wherein the tumour is likely to be resistant to treatment with the modulator of the HER signalling pathway if EGR4-S expression is at or below a predetermined level in the tumour sample.

As used herein the term “HER-expressing tumour” includes tumours that are positive for expression of a HER family member. In a preferred embodiment, the tumour comprises cells that are HER1, HER2, HER3 and/or HER4 positive.

HER1, epidermal growth factor receptor, is also known as ERBB; HER1; mENA; ERBB1; PIG61; NISBD2. The NCBI Gene ID for HER1 is 1956, and the UniProtKB/Swiss-Prot ID for HER1 is P00533.

HER2, erb-b2 receptor tyrosine kinase 2, is also known as EU; NGL; ERBB2; TKR1; CD340; HER-2; MLN 19; HER-2/neu. The NCBI Gene ID for HER2 is 2064, and the UniProtKB/Swiss-Prot ID for HER2 is P04626.

In another embodiment the breast cancer is a basal, Luminal A, Luminal B or normal-like breast cancer.

“Normal breast-like cancer” accounts for 5-10% of breast cancers, and is similar to luminal A disease: hormone-receptor positive (estrogen-receptor and/or progesterone-receptor positive), HER2 negative, and has low levels of the protein Ki-67, which helps control how fast cancer cells grow.

The present inventors have demonstrated in FIG. 3 that expression of the transcription factor isoform EGR4-S is regulated by lapatinib, gefitinib and erlotinib, modulators of the HER signalling pathway. This data indicates that cancer cells expressing the transcription factor isoform EGR4-S respond to modulators of the HER signalling pathway, indicating EGR4-S expression can be used as a marker of responsiveness.

The present application contemplates methods of identifying a subject with cancer who is likely to be responsive to treatment with a modulator of the HER signalling pathway

Accordingly, in another embodiment, the present invention provides a method of identifying a subject with cancer who is likely to be responsive to treatment with a modulator of the HER signalling pathway, wherein the method comprises;
a) providing a sample of the subject;
b) detecting the level of expression of EGR4-S in the sample of the subject, and
c) identifying the subject as being likely to be responsive to treatment with the modulator of the HER signalling pathway if EGR4-S expression is at or above a predetermined level in the sample of the subject, and wherein the subject is likely to be resistant to treatment with the modulator of the HER signalling pathway if EGR4-S expression is at or below a predetermined level in the sample of the subject.

As used herein, the phrase “providing a sample” refers to the step of obtaining a sample of the individual (e.g. cells or tumour by way of biopsy or otherwise, or blood), and/or refers to the step of receiving a sample that has previously been obtained from the individual.

The present inventors have demonstrated that directly knocking down the expression of EGR4-S in HER2+ breast cancer cells resulted in significantly reduced cancer cell growth, and that EGR4-S expression could be increased or decreased in direct relation to components of the signalling pathway such as Ras, pHER2 and pAKT levels. The identification of EGR4-S as a downstream effector of the HER pathway and it's absence from normal human tissues, further make EGR4-S an attractive target for cancer therapy.

Accordingly, in another embodiment, the present invention provides a method as described herein, further comprising administering to a subject identified as being likely to be responsive to treatment with the modulator of the HER signalling pathway a therapeutically effective amount of the modulator of the HER signalling pathway.

As used herein, the terms “effective amount”, “pharmaceutically effective amount” and “therapeutically effective amount” are used interchangeably and include an amount of a compound or agent (e.g. a modulator of the HER signalling pathway) to provide the desired biological result. That result may be reduction and/or alleviation of the signs, symptoms, or causes of a disease, e.g. cancer, or any other desired alteration of a biological system (e.g. modulation of the levels molecules downstream of EGR4-S, such as those discussed above). An appropriate therapeutic amount in any subject may be determined by one of ordinary skill in the art using routine experimentation. A therapeutic response, benefit or improvement need not be complete ablation of any one, most or all symptoms, complications, consequences or underlying causes associated with the disorder or disease. Thus, In some embodiments, a satisfactory endpoint is achieved when there is a transient, medium or long term, incremental improvement in a subject's condition, or a partial reduction in the occurrence, frequency, severity, progression, or duration, or inhibition or reversal, of one or more associated adverse symptoms or complications or consequences or underlying causes, worsening or progression (e.g., stabilizing one or more symptoms or complications of the condition, disorder or disease), of the disorder or disease, over a duration of time (hours, days, weeks, months, and so forth). An “effective amount” will vary from subject to subject, depending on the age and general condition of the individual and with the factors such as the particular condition being treated or prevented, the duration of the treatment, previous treatments and the nature and pre-existing duration of the condition.

In one embodiment, the present invention provides a method as described herein, wherein the therapeutically effective amount of a compound or agent (e.g. a modulator of the HER signalling pathway) is administered in two or more doses.

In another embodiment, the present invention provides a method as described herein, wherein the therapeutically effective amount of a compound or agent (e.g. a modulator of the HER signalling pathway) is administered daily, weekly, biweekly, bimonthly, and or quarterly.

In a further embodiment, the present invention provides a method as described herein, wherein the subject administered with a therapeutically effective amount of a compound or agent (e.g. a modulator of the HER signalling pathway) is treated before, during, after, or simultaneously with one or more additional therapies for the treatment of the cancer.

In a further embodiment, the present invention provides a method as described herein, wherein the therapeutically effective amount of a compound or agent (e.g. a modulator of the HER signalling pathway) is administered orally, intravenously, intramuscularly, subcutaneously, topically or a combination thereof.

In a further embodiment, the present invention provides a method as described herein, wherein the therapeutically effective amount of a compound or agent (e.g. a modulator of the HER signalling pathway) is formulated as a composition further comprising one or more pharmaceutically acceptable excipients.

In one embodiment, the present invention provides a method of treating or preventing a HER pathway activated cancer; wherein the method comprises;
a) providing a sample of the subject;
b) detecting the level of expression of EGR4-S in the sample of the subject;
c) identifying the subject as being likely to be responsive to treatment with the modulator of the HER signalling pathway if EGR4-S expression is at or above a predetermined level in the sample of the subject; and
d) administering to a subject identified as being likely to be responsive to treatment with the modulator of the HER signalling pathway a therapeutically effective amount of the modulator of the HER signalling pathway.

As used herein, the term “treatment” or “treating” includes the application or administration of a therapeutic agent, e.g. a modulator of the HER signalling pathway a compound disclosed herein (alone or in combination with another pharmaceutical agent), to a patient, or application or administration of a therapeutic agent to an isolated tissue or cell from a patient who has a disease or a condition described herein (e.g. cancer, including a HER2+ cancer, a HER2+ and EGR4S+ cancer etc.), a symptom of a disease, or of a condition contemplated herein or the potential to develop a disease or a condition contemplated herein, with the purpose to cure, heal, alleviate, relieve, alter, remedy, ameliorate, improve or affect a disease or condition contemplated herein, the symptoms of a disease or a condition contemplated herein or the potential to develop a disease or a condition contemplated herein. Such treatments may be specifically targeted, or may be broad spectrum. For example, an effective amount of the therapeutic agent may reduce the number of cancer cells; reduce the tumour size; inhibit (i.e., slow to some extent and preferably stop) cancer cell infiltration into peripheral organs; inhibit (i.e., slow to some extent and preferably stop) tumour metastasis; inhibit, to some extent, tumour growth; and/or relieve to some extent one or more of the symptoms associated with the cancer. To the extent the agent may prevent growth and/or kill existing cancer cells, it may be cytostatic and/or cytotoxic. The effective amount may extend progression free survival (e.g. as measured by Response Evaluation Criteria for Solid Tumours, RECIST, or CA-125 changes), result in an objective response (including a partial response, PR, or complete response, CR), improve survival (including overall survival and progression free survival) and/or improve one or more symptoms of cancer (e.g. as assessed by FOSI). Most preferably, the therapeutically effective amount of the agent is effective to improve progression free survival (PFS) and/or overall survival (OS).

Examples of treatments include chemotherapeutic agents including alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin I and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g., calicheamicin, especially calicheamicin gamma1I and calicheamicin omegaII (see, e.g., Nicolaou et al., Angew. Chem Intl. Ed. Engl., 33: 183-186 (1994)); CDP323, an oral alpha-4 integrin inhibitor; dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, catminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), liposomal doxorubicin TLC D-99 (MYOCET®), peglylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; anti-adrenals such as aminoglutethimide, mitotane, trilostane; folic acid replenisher such as frolinic acid; aceglatone; aldophosphamide glycoside; am inolevulinic acid; eniluracil; amsacrine; bestrabucil; bisantrene; edatraxate; defofamine; demecolcine; diaziquone; elfornithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; dacarbazine; mannomustine; mitobronitol; mitolactol; pipobroman; gacytosine; arabinoside (“Ara-C”); thiotepa; taxoid, e.g., paclitaxel (TAXOL®), albumin-engineered nanoparticle formulation of paclitaxel (ABRAXANE™), and docetaxel (TAXOTERE™); chloranbucil; 6-thioguanine; mercaptopurine; methotrexate; platinum agents such as cisplatin, oxaliplatin, and carboplatin; vincas, which prevent tubulin polymerization from forming microtubules, including vinblastine (VELBAN®), vincristine (ONCOVIN®), vindesine (ELDISINE®, FILDESIN®), and vinorelbine (NAVELBINE®); etoposide (VP-16); ifosfamide; mitoxantrone; leucovorin; novantrone; edatrexate; daunomycin; am inopterin; ibandronate; topoisomerase inhibitor RFS 2000; difluorometlhylornithine (DMFO); retinoids such as retinoic acid, including bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); BAY439006 (sorafenib; Bayer); SU-11248 (Pfizer); perifosine, COX-2 inhibitor (e.g. celecoxib or etoricoxib), proteosome inhibitor (e.g. PS341); bortezomib (VELCADE®); CCI-779; tipifarnib (R11577); orafenib, ABT510; Bcl-2 inhibitor such as oblimersen sodium (GENASENSE®); pixantrone; EGFR inhibitors (see definition below); tyrosine kinase inhibitors (see definition below); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin,

Examples of treatments also include anti-hormonal agents or endocrine therapeutics which act to regulate, reduce, block, or inhibit the effects of hormones that can promote the growth of cancer. They may be hormones themselves, including, but not limited to: anti-estrogens with mixed agonist/antagonist profile, including, tamoxifen (NOLVADEX®), 4-hydroxytamoxifen, toremifene (FARESTON®), idoxifene, droloxifene, raloxifene (EVISTA®), trioxifene, keoxifene, and selective estrogen receptor modulators (SERMs) such as SERM3; pure anti-estrogens without agonist properties, such as fulvestrant (FASLODEX®), and EM800 (such agents may block estrogen receptor (ER) dimerization, inhibit DNA binding, increase ER turnover, and/or suppress ER levels); aromatase inhibitors, including steroidal aromatase inhibitors such as formestane and exemestane (AROMASIN®), and nonsteroidal aromatase inhibitors such as anastrazole (ARIMIDEX®), letrozole (FEMARA®) and aminoglutethimide, and other aromatase inhibitors include vorozole (RIVISOR®), megestrol acetate (MEGASE®), fadrozole, and 4(5)-imidazoles; lutenizing hormone-releaseing hormone agonists, including leuprolide (LUPRON® and ELIGARD®), goserelin, buserelin, and tripterelin; sex steroids, including progestines such as megestrol acetate and medroxyprogesterone acetate, estrogens such as diethylstilbestrol and premarin, and androgens/retinoids such as fluoxymesterone, all transretionic acid and fenretinide; onapristone; anti-progesterones; estrogen receptor down-regulators (ERDs); anti-androgens such as flutamide, nilutamide and bicalutamide; and pharmaceutically acceptable salts, acids or derivatives of any of the above; as well as combinations of two or more of the above.

Examples of treatments further include an antimetabolite chemotherapeutic agent, which is an agent which is structurally similar to a metabolite, but can not be used by the body in a productive manner. Many antimetabolite chemotherapeutic agents interfere with the production of the nucleic acids, RNA and DNA. Examples of antimetabolite chemotherapeutic agents include gemcitabine (GEMZAR®), 5-fluorouracil (5-FU), capecitabine (XELODA™), 6-mercaptopurine, methotrexate, 6-thioguanine, pemetrexed, raltitrexed, arabinosylcytosine ARA-C cytarabine (CYTOSAR-U®), dacarbazine (DTIC-DOME®), azocytosine, deoxycytosine, pyridmidene, fludarabine (FLUDARA®), cladrabine, 2-deoxy-D-glucose etc. The preferred antimetabolite chemotherapeutic agent is gemcitabine.

As used herein the term “treatment” includes therapeutic treatment as well as prophylactic treatment (either preventing the onset of a disorder or a symptom of a disorder (including an age-related change) altogether or delaying the onset of a symptom of a disorder (including an age-related change), or a preclinically evident stage of a disorder in an individual). In some embodiments, the term “treatment” or “treating” refers to an action that occurs while an individual is suffering from the specified cancer, which reduces the severity of the cancer or the symptoms of the cancer, and/or retards or slows the progression of the cancer. For instance, in some embodiments, “treatment” or “treat” refers to a 5%, 10%, 25%, 50% or 100% decrease in the rate of cell growth and/or progress of a tumour. In other embodiments, “treatment” refers to a 5%, 10%, 25%, 50% or 100% decrease in determined tumour burden (i.e., number of cancerous cells present in the individual, and/or the size of the tumour). In yet other embodiments, “treatment” refers to a 5%, 10%, 25%, 50% or 100% decrease in any physical symptom(s) of a cancer. In yet other embodiments, “treatment” refers to a 5%, 10%, 25%, 50% or 100% increase in the general health of the individual, as determined by any suitable means, such as cell counts, assay results, or other suitable means.

In one embodiment treating includes inhibiting cancer cell growth.

The term “prevention” includes either preventing the onset of a disorder or a symptom of a disorder altogether or delaying the onset of disorder or a symptom of a disorder, or a preclinically evident stage of a disorder in an individual. This includes prophylactic treatment of those at risk of developing a disease, such as a cancer, for example. “Prophylaxis” is another term for prevention. As used herein term “prevent”, “preventing” or “prevention,” includes avoiding or delaying the onset of symptoms associated with a disease or condition in a subject that has not developed such symptoms at the time the administering of a modulator of the HER signalling pathway or anti-cancer treatment commences.

As used herein, the term “HER-pathway activated cancer” includes a cancer that is caused or promoted in any way by a mutation in one of the HER proteins, such as EGFR gene fusion, a EGFR kinase domain duplication, a ErbB-2 gene fusion, a ErbB-2 mutation, a NRGI gene fusion, a ErbB-3 mutation, and/or a ErbB-4 fusion and the like. The HER-pathway activated cancer may be indicated by the presence of a EGFR gene fusion, a EGFR kinase domain duplication, & ErbB-2 gene fusion, & ErbB-2 mutation, a NRGI gene fusion, a ErbB-3 mutation, and/or a ErbB-4 fusion. The HER-pathway activated cancer may be resistant to osimertinib, gefitinib, afatinib, and/or erlotinib, as described herein. In some embodiments, the HER-pathway activated cancer has an EGFR mutation, or an ErbB-2 mutation, where the EGFR and/or ErbB-2 mutation is indicated phenotypically, for example, by histopathology, imaging, tumour growth, DNA analysis, RNA analysis or other diagnostic means, as described herein. In some embodiments, a mutation can be identified from general biological samples, e.g., from blood, tissue, urine, and the like, by detecting, e.g., downstream biochemical markers, metabolism markers, circulating RNA, or circulating DNA, and the like, that are indicative of the specific mutations. In some embodiments, the mutations can be tested using, e.g., direct tumour biopsy or liquid biopsy using ctDNA or CTCs.

TKI drugs are an effective targeted therapy for HER-overexpressing cancer, however, resistance still develops in many patients (Berns et al., 2007; Lin et al., 2014). The present inventors have demonstrated in FIG. 12 that the development of TKI drug resistance in breast cancer cells is linked to the responsiveness of EGR4-S to treatment. In particular, once drug resistance developed, EGR4-S expression remained constant and unchanging, despite further drug treatment. These results suggest EGR4 dysregulation can be used as an indicator of drug responsiveness to determine whether the treatment is effective. For example, when the level of EGR4-S is maintained in tumours despite continued administration of drugs, treatment (e.g. targeted treatment) can be discontinued, and/or non-targeted therapies used.

The term “resistance” as used herein, refers to a condition wherein a cancer that was sensitive to the effects of a modulator of the HER signalling pathway becomes non-responsive or less-responsive over time to the effects of that modulator of the HER signalling pathway.

In one embodiment, the method further comprises administering to a subject identified as being likely to be resistant to treatment with the modulator of the HER signalling pathway a therapeutically effective amount of the a non-HER signalling pathway anti-cancer therapy.

The present inventors have also demonstrated that the level of expression of HSF1 can be used in the methods described herein.

For example, Example 3 demonstrates EGR4-S is regulated by HSF1 in normal and oncogenically transformed cells, and EGR4-S expression decreases with increasing HSF1 expression in oncogenically transformed breast cells. Furthermore, FIG. 3 demonstrates that expression of the transcription factor isoform EGR4-S is regulated by lapatinib, gefitinib and erlotinib, modulators of the HER signalling pathway. Accordingly, HSF-1 can be used as a marker of responsiveness, either alone or in combination with EGR4-S.

Example 7 demonstrates increased HSF1 expression is a marker of increased molecular stress in cancer cells and increased EGR4-S expression is a marker of decreased molecular stress in cancer cells. Accordingly, HSF-1 expression can be used as a marker of responsiveness, either alone or in combination with EGR4-S. HSF-1 expression can also be used as a marker of molecular stress, either alone or in combination with EGR4-S.

Importantly the present inventors have demonstrated in FIG. 5 that increasing HSF-1 expression results in slower cancer cell growth. FIGS. 6 and 7 demonstrate that HSF1 expression is associated with hallmarks of metastatic cancer. Accordingly, HSF-1 expression can be used as a marker of metastasis, either alone or in combination with EGR4-S.

Without wishing to be bound by theory, the present inventors propose that reducing molecular stress might enable cancer cells to be more responsive to treatment. For example, it has been demonstrated that stress accelerates cancer progression (Calderwood and Gong, 2011; Sims et al., 2011; O'Callaghan-Sunol 2006; Khaleque et al., 2008) and drugs that activate stress such as HSP90 inhibitors (AUY-922, 17AAG, Geldanamycin) have limited effectiveness in cancer and even cause metastasis. Accordingly, avoiding the use of drugs that activate stress in EGR4-S positive tumours can decrease metastatic changes demonstrated herein that are associated with reducing EGR4-S expression.

In another embodiment, the present invention provides a method as described herein, further comprising detecting the level of expression of HSF1 in the sample of the subject.

HSF1, heat shock transcription factor 1, is also known as HSTF1. The NCBI Gene ID for HSF1 is 3297, and the UniProtKB/Swiss-Prot ID for HSF1 is Q00613.

In another embodiment, the present invention provides a method as described herein, wherein the subject is likely to be resistant to treatment with the modulator of the HER signalling pathway if EGR4-S expression is at or below a predetermined level in the sample of the subject, and HSF1 expression is at or above a predetermined level in the sample of the subject.

In another embodiment, the present invention provides a method as described herein, wherein the subject is likely to be responsive to treatment with the modulator of the HER signalling pathway if EGR4-S expression is at or above a predetermined level in the sample of the subject, and HSF1 expression is at or below a predetermined level in the sample of the subject.

As discussed above, TKI drugs are an effective targeted therapy for HER-overexpressing cancer, however, resistance still develops in many patients. The present inventors have demonstrated in FIG. 12 that the development of TKI drug resistance in breast cancer cells is linked to the responsiveness of EGR4-S to treatment. In particular, once drug resistance developed, EGR4-S expression remained constant and unchanging, despite further drug treatment.

This demonstrates that EGR4-S can be a good indicator of drug responsiveness to determine whether the treatment is effective. For example, when the level of EGR4-S is maintained in tumours despite continued administration of drugs, treatment (e.g. targeted treatment) can be discontinued, and/or non-targeted therapies used.

Accordingly, in one embodiment, the present invention provides a method of monitoring the stress of a cancer during treatment with a modulator of the HER signalling pathway, wherein the method comprises detecting the level of expression of HSF-1 and EGR4-S in a sample of a subject with cancer, wherein:
a) when HSF-1 expression is at or above a predetermined level in the sample of the subject and EGR4-S expression is at or below a predetermined level in the sample of the subject, the stress of a cancer of the subject is increased;
b) when HSF-1 expression is at or below a predetermined level in the sample of the subject and EGR4-S expression is at or above a predetermined level in the sample of the subject, the stress of a cancer of the subject is decreased; or
c) when HSF-1 expression is at or above a predetermined level in the sample of the subject and EGR4-S expression is at or above a predetermined level in the sample of the subject, the stress of a cancer of the subject is increased.

As used herein the term “stress of a cancer” refers to a phenotypic state or stage of a cancer, e.g. a tumour, including phenotypes characterised by expression of molecular markers of stress, including heat shock proteins, heat shock factors etc.

In one embodiment, the method further comprises administering to a subject identified as being likely to be resistant to treatment with the modulator of the HER signalling pathway a therapeutically effective amount of the a non-HER signalling pathway anti-cancer therapy.

Accordingly, in one embodiment the present invention provides a method of monitoring resistance to treatment with a modulator of the HER signalling pathway during treatment with a modulator of the HER signalling pathway, wherein the method comprises detecting the level of expression of HSF-1 and EGR4-S in a sample of a subject with cancer, wherein when HSF-1 expression is at or above a predetermined level in the sample of the subject and EGR4-S expression is at or above a predetermined level in the sample of the subject, the subject likely to be resistant to treatment with the modulator of the HER signalling pathway.

In one embodiment, wherein when HSF-1 expression is at or above a predetermined level in the sample of the subject and EGR4-S expression is at or above a predetermined level in the sample of the subject, the subject is likely to be resistant to treatment with the modulator of the HER signalling pathway.

In another embodiment, a subject likely to be resistant to treatment with the modulator of the HER signalling pathway is administered a HSP modulating agent, for example, a HSP inhibitor. In one embodiment the HSP modulating agent is 17-AAG or genetisnib.

In another embodiment the present invention provides a method of monitoring the stress of a cancer during treatment with a modulator of the HER signalling pathway, wherein the method comprises detecting the level of expression of HSF-1 and EGR4-S in a sample of a subject with cancer, wherein when HSF-1 expression is at or above a predetermined level in the sample of the subject and EGR4-S expression is at or above a predetermined level in the sample of the subject, the stress of a cancer of the subject is increased.

In another embodiment, a subject with a cancer with increased stress is administered a HSP modulating agent, for example, a HSP inhibitor. In one embodiment the HSP modulating agent is 17-AAG or genetisnib.

In another embodiment, the present invention provides a method as described herein, wherein the method further comprises detecting the level of expression of HER1 and/or HER2 in a sample of the subject.

Accordingly, the relative proportion, levels or ratios of more than one of EGR4S, HSF1, HER1, HER2 etc. be used.

In a preferred embodiment, the ratio of expression of EGR4S and HSF is determined.

In another embodiment, the present invention provides a method as described herein, wherein the subject is likely to be responsive to treatment with the modulator of the HER signalling pathway if EGR4-S, and HER1 and/or HER2, is at or above a predetermined level in the sample of the subject.

In another embodiment, the present invention provides a method as described herein, wherein the predetermined level is the amount or level of EGR4-S, HSF1, HER1 and/or HER2 in a control or reference sample.

In another embodiment, the present invention provides a method as described herein, wherein the control or reference sample is a sample of cells obtained from: a normal healthy individual or individuals; a patient or patients with an analogous cancer; normal tissue adjacent to tumour tissue of the same subject; tumour cells of the same subject provided at a different time; or a cell line.

In another embodiment, the present invention provides a method as described herein, wherein the modulator of the HER signalling pathway is an EGFR inhibitor.

In another embodiment, the present invention provides a method as described herein, wherein the modulator of the HER signalling pathway lapatinib, erlotinib, gefitinib, cetuximab, osimertinib, panitumumab, or neratinib.

In another embodiment, the present invention provides a method as described herein, wherein the step of detecting the level of expression of EGR4-S in the sample comprises using PCR or RT-PCR.

In another embodiment, the present invention provides a method as described herein, wherein the step of detecting the level of expression of EGR4-S in the sample comprises using PCR or RT-PCR across the EGR4 exon 1/exon 2 splice site.

In another embodiment, the present invention provides a method as described herein, wherein the step of detecting the level of expression of EGR4-S in the sample comprises using PCR or RT-PCR across the EGR4 exon 1/exon 2 splice site, and using PCR or RT-PCR amplification of EGR4 exon 1 and/or exon 2.

In another embodiment, the present invention provides a method as described herein, wherein the step of detecting the level of expression of EGR4-S in the sample comprises using FISH.

In another embodiment, the present invention provides a method as described herein, wherein the step of detecting the level of expression of EGR4-S in the sample comprises using an immunohistochemical assay for EGR4-S polypeptide expression.

In another embodiment, the present invention provides a method as described herein, wherein the step of detecting the level of expression of EGR4-S in the sample comprises using histology, ELISA, an ELISA-like assay, Western Blot, and/or flow cytometry to assay for EGR4-S polypeptide expression.

In another embodiment, the present invention provides a method as described herein, wherein the cancer is a solid tumour.

In another embodiment, the present invention provides a method as described herein, wherein the solid tumour is breast cancer tumour or lung cancer.

In another embodiment, the present invention provides a method as described herein, wherein the cancer is selected from ovarian cancer, peritoneal cancer, fallopian tube cancer, metastatic breast cancer (MBC), non-small cell lung cancer (NSCLC), prostate cancer, and colorectal cancer.

In another embodiment, the present invention provides a method as described herein, wherein the method is performed prior to, concurrent with and/or following treatment with a modulator of the HER signalling pathway.

In another embodiment, the present invention provides a method as described herein, wherein the modulator of the HER signalling pathway is administered by at least one route selected from orally, intravenously, intramuscularly, subcutaneously, topically or a combination thereof.

In another embodiment, the present invention provides a method as described herein, wherein the subject likely to be resistant to treatment with the modulator of the HER signalling pathway has metastatic cancer.

In another embodiment, the present invention provides a method as described herein, further comprising administering to a subject identified as being likely to be resistant to treatment with the modulator of the HER signalling pathway a therapeutically effective amount of the a non-HER signalling pathway anti-cancer therapy.

In another embodiment, the present invention provides a method of treating or preventing a HER pathway activated cancer; wherein the method comprises administering to a subject a therapeutically effective amount of a modulator of EGR4-S activity.

In one embodiment the present invention provides a method of treating or preventing growth of a tumour of a HER pathway activated cancer; wherein the method comprises administering to a subject a therapeutically effective amount of a modulator of EGR4-S activity.

In one embodiment, the therapeutically effective amount of a modulator of EGR4-S activity is a pharmaceutical composition comprising a therapeutically effective amount of a modulator of EGR4-S activity. As used herein, the term “pharmaceutical composition” refers to a mixture of at least one compound contemplated herein with a pharmaceutically acceptable carrier. The pharmaceutical composition facilitates administration of the modulator to a patient or subject.

In another embodiment, the present invention provides a method as described herein, wherein the modulator of EGR4-S activity is selected from the group consisting of an EGFR inhibitor, an siRNA, and a shRNA.

As described above, in another embodiment, the present invention provides a method as described herein, wherein the sample of the subject is selected from a sample of: tumour cells, whole blood, serum, plasma, a sample comprising cell-free DNA and/or cell-free RNA, or a combination thereof.

In another embodiment, the present invention provides a method as described herein, wherein the sample is blood serum.

In one embodiment the present invention provides a method of treating a patient with cancer by administering a modulator of the HER signalling pathway, wherein the patient has a cancer with EGR4-S expression at or above a predetermined level in the sample of the subject.

In one embodiment the present invention provides a method of treating a patient with cancer by administering modulator of the HER signalling pathway, wherein the patient has a cancer with HSF1 expression at or below a predetermined level in the sample of the subject.

In one embodiment the present invention provides a method of treating a patient with cancer by administering a therapy that is not a modulator of the HER signalling pathway, wherein the patient has a cancer with EGR4-S expression at or below a predetermined level in the sample of the subject.

In one embodiment the present invention provides a method of treating a patient with cancer by administering a therapy that is not a modulator of the HER signalling pathway, wherein the patient has a cancer with HSF1 expression at or above a predetermined level in the sample of the subject.

### EXAMPLES

### Example 1—Materials and Methods

Generation and Sources of Plasmid Constructs

Plasmid constructs were generated as described previously (Nguyen et al., 2013). All expression vector sequences were confirmed by DNA sequencing (Micromon DNA Sequencing Facility, Monash University).

Cell Lines and Cell Cultures

The MCF10A cell line was obtained from the A.T.C.C. (Manassas, VA, U.S.A.) and cultured as described previously (Debnath et al., 2003). T47D cells, SkBr3 cells, Hs578T and HEK-293T cells were cultured as described by Nguyen et al (2013). MDA-361, ZR75-1, BT-474, MDA-453, MDA-468, MDA-231, BT549 and MCF7 cells were cultured in Dulbecco's Modified Eagle's medium supplemented with 10% (v/v) fetal bovine serum and 1% (w/v) penicillin/streptomycin. In addition, MCF7 growth media was supplemented with 10 ng/ml insulin. All stable cell lines were generated by retroviral or lentiviral transduction as per Debnath et al. (2003). Viral stocks were generated by transient transfection of appropriate viral packaging vectors into the HEK-293T cell line as described by Lang and colleagues (2012). Drugs/compounds used for treating the cells were purchased from commercial sources, including Lapatinib, Erlotinib, Gefinitib and AUY922 from Sigma, and Sulfurophane from Santa Cruz Biotechnology. HSF1-targeted shRNAmir (microRNA-adapted short hairpin RNA) vectors were constructed as described previously (Lang et al., 2012). EGR4-targeted shRNAmir were purchased from Millenium Science.

Microarray Analysis

Cells were grown in 10 cm cell culture dishes to 50-70% confluency and total RNA was extracted using a Qiagen RNA extraction kit (Qiagen, California, USA) according to the manufacturer's instructions. RNA was diluted to 50 ng/μl and submitted to Agilent Technologies (The Ramaciotti Center, New South Wales, Australia) for microarray processing. Metacore™ bioinformatics software was used to analyse the resulting data (GeneGo, Thompson Reuters, USA).

Seahorse Analysis

Experiments to measure cellular glycolysis were performed using a Seahorse XF24 Analyser (Agilent, USA) and Glycolysis Stress Test kit (Agilent, USA). The optimal cell seeding number was determined by initial titration experiments. The Seahorse sensor cartridge was hydrated for 24 hours in XF Calibrant liquid prior to each experiment. Immediately before the experiment, each plate was loaded with the supplied Glycolysis kit compounds according to the manufacturer's specifications. After seeding 80,000 cells/well, the cells were allowed to adhere to the plate for 1 hr before loading both the sensor plate and cell culture plate into the Seahorse machine for analysis. At the completion of the experiment, the data from each sample was analysed using “Wave v2.3” software.

Western Blot Analysis and Antibodies

Generation of protein lysates from cells was performed according to a previously published protocol (Price et al., 2005). Equal concentrations of protein lysates were combined with loading buffer (Invitrogen) and sample reducing agent (Invitrogen), denatured for 5 minutes at 95° C. then separated through gel electrophoresis in a 4-to-12% Bis-Tris NuPAGE gel (Invitrogen) with 1×MES buffer (Invitrogen) and antioxidant (Invitrogen). Following separation, the proteins were electrophoretically transferred onto nitrocellulose membranes using the iBlot western detection stack/iBlot dry blotting system (Thermofisher) for seven minutes at 23V. All membranes were then processed at room temperature overnight using the iBlot Western system (Thermofisher) containing primary antibodies, iBind wash solution and horseradish peroxidase-conjugated secondary antibodies. Blots were developed using Clarity Western ECL (Bio-RAD), and images were captured using a Fusion FX imaging machine (Vilber). All antibodies were purchased from commercial sources and included, anti-HSF1 (catalogue number SPA-901), anti-HSP27 (catalogue number SPA-800) and anti-HSP90a (catalogue number SPA-835) antibodies from Enzo Life Sciences; anti-HSP105/110 (catalogue number Sc-6241) antibody from Santa Cruz Biotechnology; anti-Ha-Ras (catalogue number 05-775) antibody from Merck Millipore; anti-actin (catalogue number MS-1295-P0) and anti-HSP70i (catalogue number MS-482-P0) antibodies from Thermo Scientific; anti-p53 (catalogue number 51-9002046) from BD Pharmingen; anti-pHER2/ErbB2 (catalogue number Ab53290), anti-pAkt (catalogue number MISCGENLAB), anti-pEGFR catalogue number SCZSC-12351), anti-total EGFR catalogue number SCZSC-120) from Abcam, anti-total Akt (catalogue number 9272) and anti-HER2/ErbB2 (catalogue number Ab134182) from Cell Signalling, anti-EGR4 (catalogue number AV38090) (Sigma), anti-EGR4 (catalogue number PA5-50496) (Thermofisher), and anti-EGR4 (catalogue number SC133540) (Santa Cruz).

Three Dimensional Adhesion-Independent Clonogenic Growth Assay

MCF10A cells were grown and analysed with this assay as described by Nguyen et al (2013).

Cell Proliferation assay using xCELLigence System

xCELLigence experiments were performed using the Real-Time Cell Analyzer instrument according to manufacturers' instructions (ACEA Biosciences, San Diego, CA). The optimal cell seeding number for each cell line (B-T549, MDA-MB-231, HCC-1143 and HCC-1937) was determined by initial titration experiments. After seeding 10,000 cells/well, plates were loaded onto the machine and automated cell index readings were taken every 30 minutes for 120 hours. Cells were treated with the compounds approximately 24 hours after seeding, when the cells were in the log growth phase. For each assay, cells were treated with fresh media as control or different concentrations of drug/compound: Lapatinib (0.0125, 0.025, 0.05, 0.1, 0.125, 0.2, 0.25, 0.5, 1, 2 μM), Erlotinib (0.125, 0.2, 0.25, 0.5 μM), Gefitinib (0.125, 0.2, 0.25, 0.5 μM), AUY922(12.5, 25, 50, 100, 200 nM), and Sulfurophane (1.25, 2.5, 5 μM). At the completion of the experiment, the cell index value was calculated for each sample using the RTCA Software Package 1.2.

Microchemotaxis Migration Assay

Cell migration was examined using 48-well microchemotaxis chamber assay (Neuro Probe, Maryland, USA) as described previously by Kouspou and Price (Kouspou and Price, 2011). The chemoattractants used in this study were Fibroblast conditioned media (FbCM) and EGF (20 ng/ml for MCF10A, 10 ng/ml for SkBr3).

Statistical Analysis

All cell biology assays were performed at least three times and the results were combined. The results presented are means±S.D. Student's t tests were conducted to determine whether the treatment group was statistically significant compared with control. Significance is represented as either *P<0.05, **P<0.01 or ***P<0.001.

### Example 2—Expression of HSF1 Causes Increased Migratory Behaviour of Breast Cancer Cells, an Increase in Disorganised Growth, Increased HSP Expression, and Decreased Glycolysis

The impact of HSF1 activation on the cell biology and oncogenicity of ‘normal’ human mammary epithelial cells was examined. MCF10a cells were transduced by retroviruses that contained vectors with either GFP as a control, wild type (WT) HSF1 or constitutively activated HSF1 (HSF1 ΔRDT) (Fujimoto et al., 2005). The MCF10a cells expressing GFP or overexpressing HSF1 (HSF1 WT or HSF1 ΔRDT) were subsequently transduced by retroviral vectors to stably express either mCherry (as a control) or the activated oncogene H-Rasv12.

FIG. 6 shows that with increased molecular stress resulting from expression of constitutively activated HSF1, oncogenically transformed breast cancer cells (MCF10a cells) were significantly more migratory (FIG. 6B) than normal cells expressing constitutively active HSF1 (FIG. 6A).

FIG. 7 shows that with increased molecular stress resulting from expression of constitutively activated HSF1, oncogenically transformed breast cancer cells (MCF10a cells) had a significant increase in disorganised growth (FIGS. 7A and B) than normal cells expressing constitutively active HSF1.

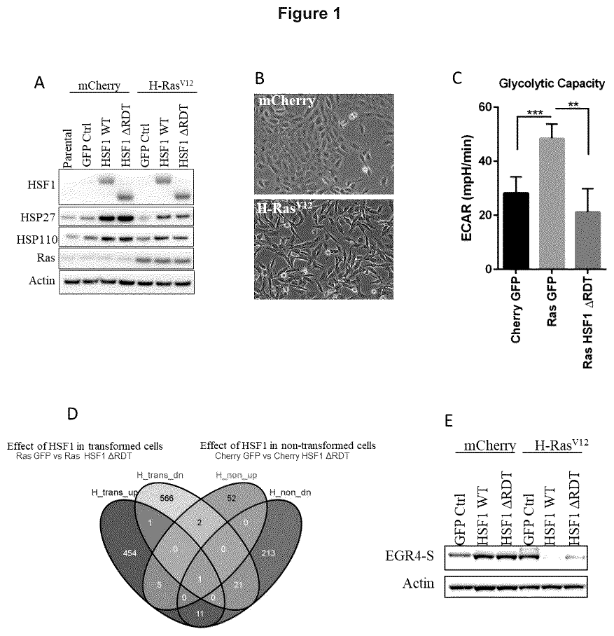

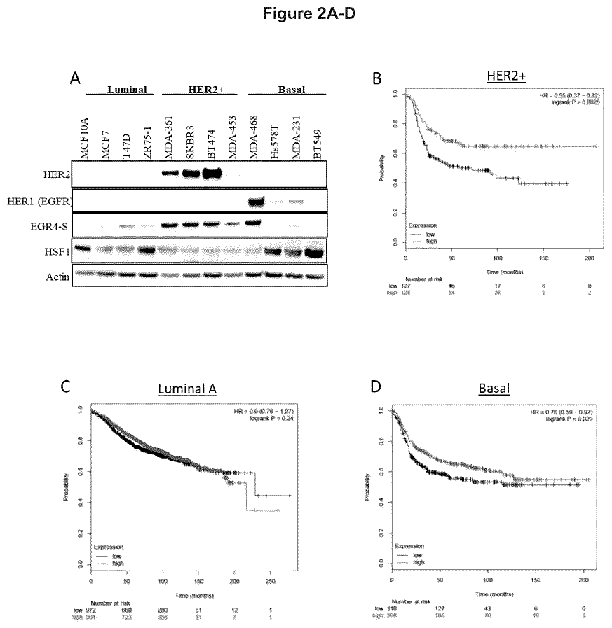

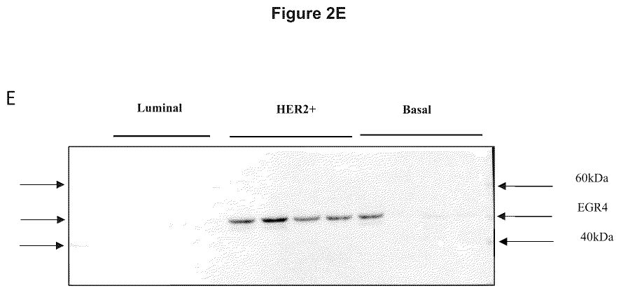

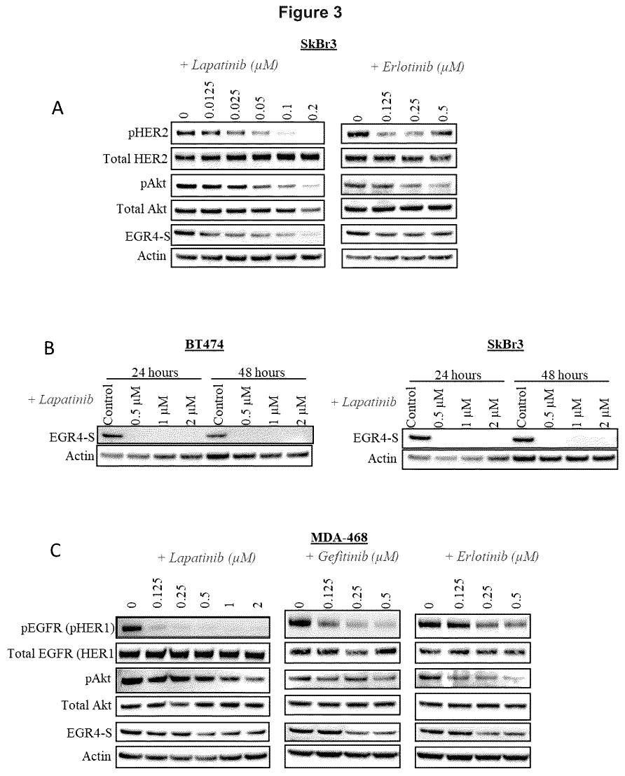

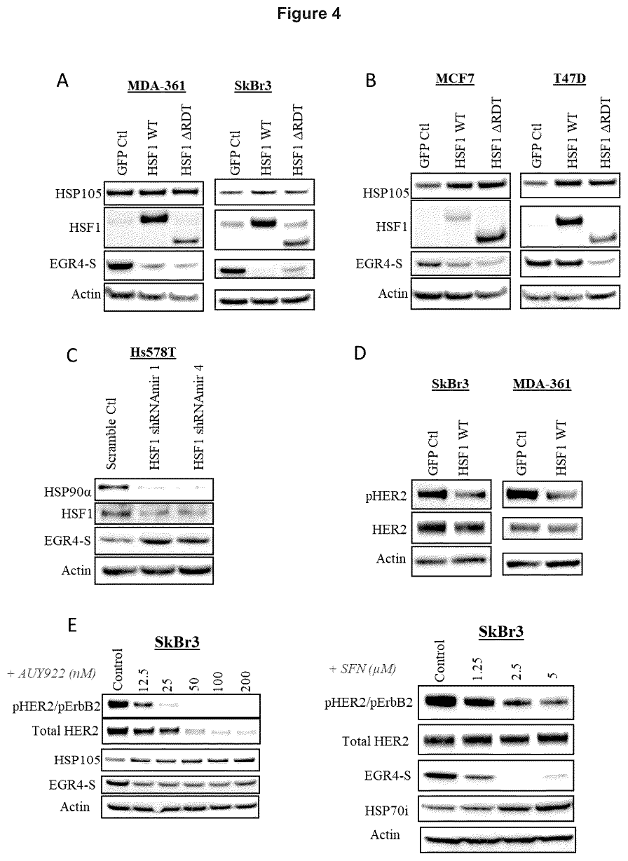

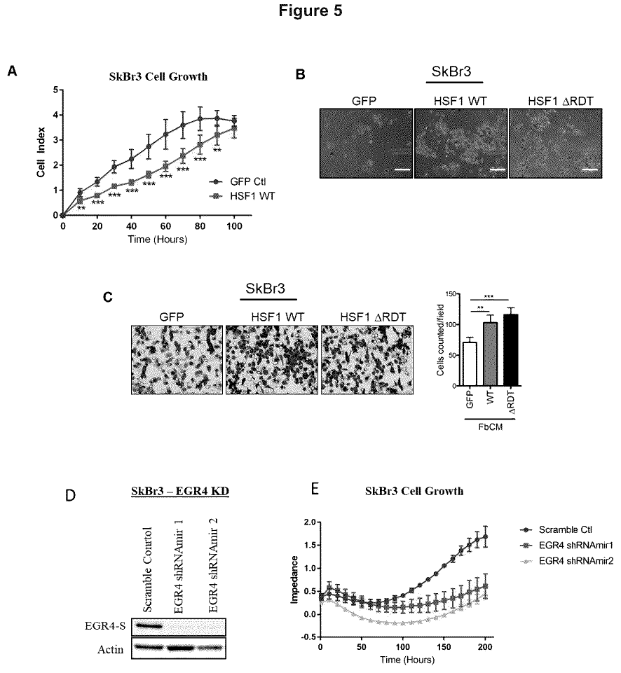

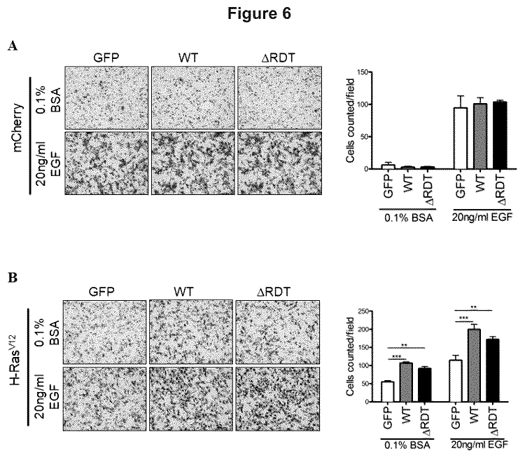

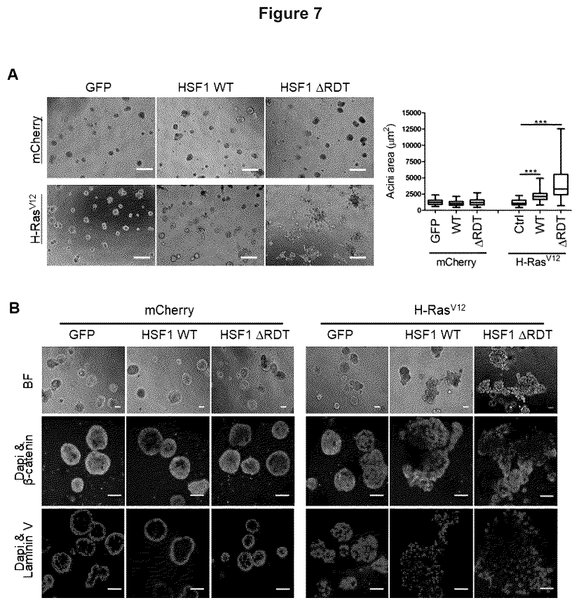

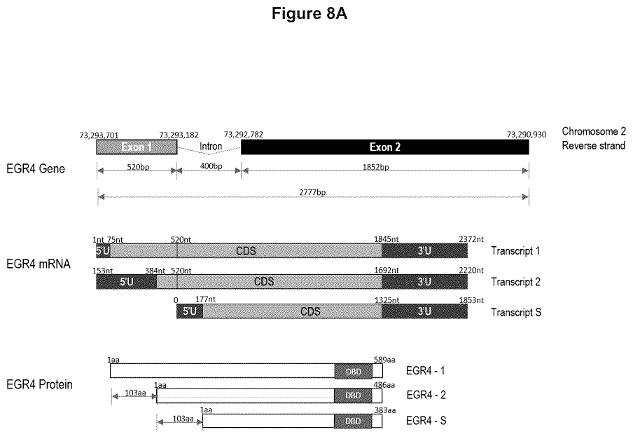

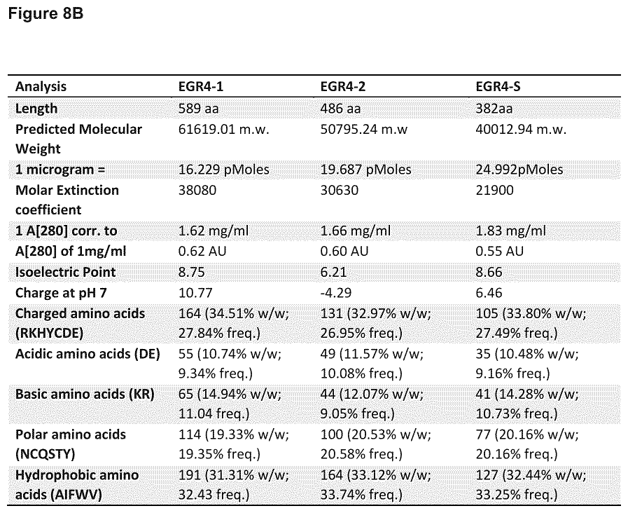

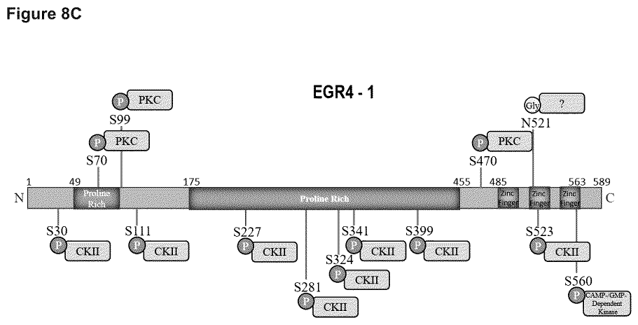

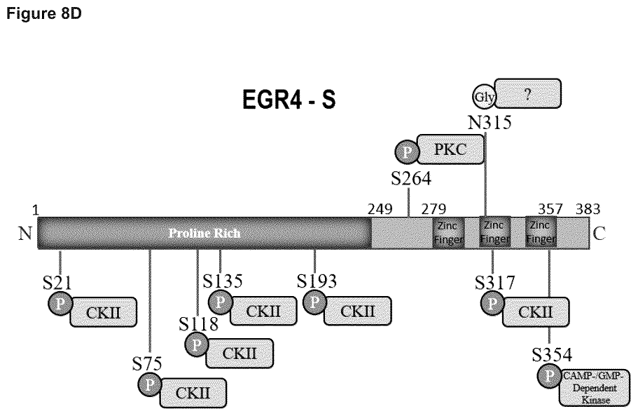

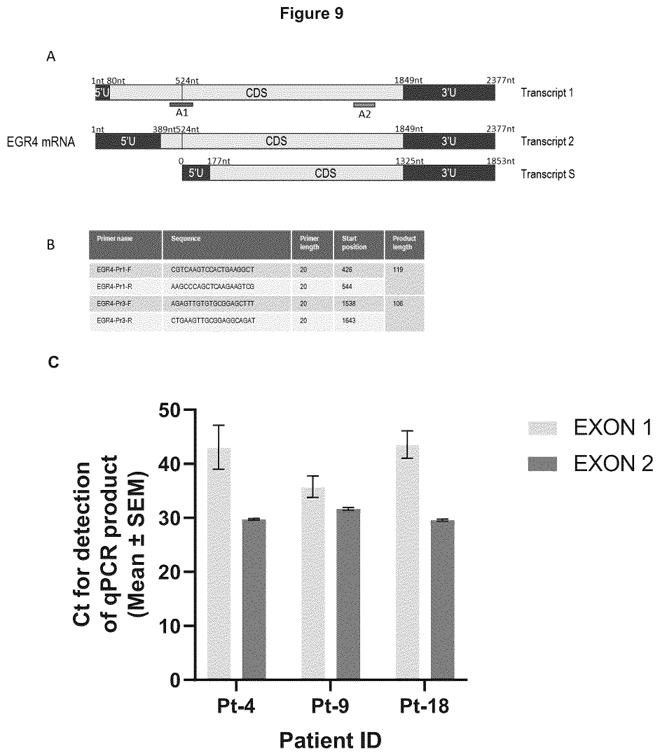

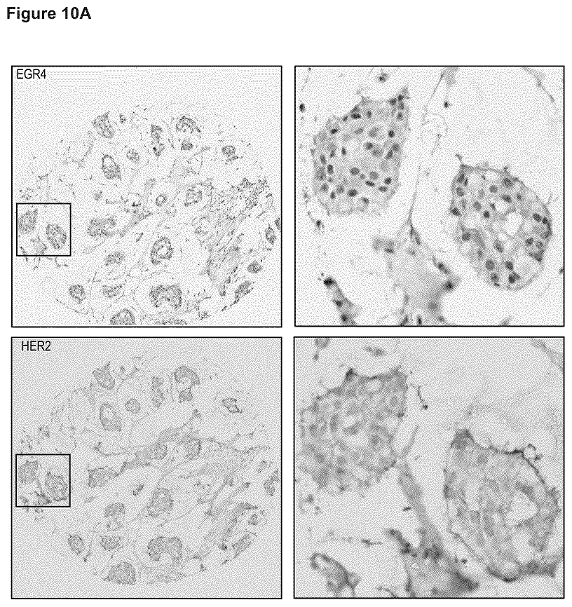

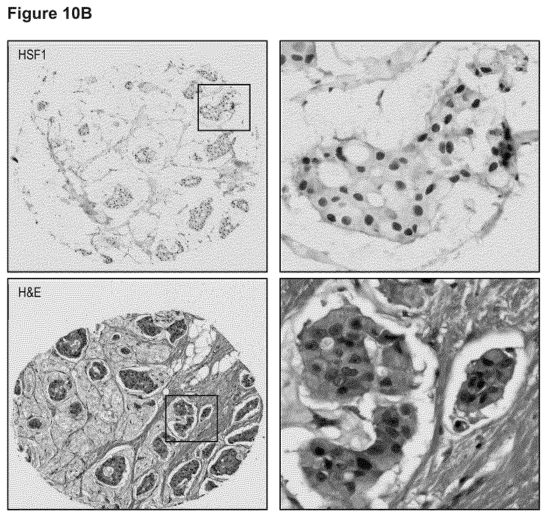

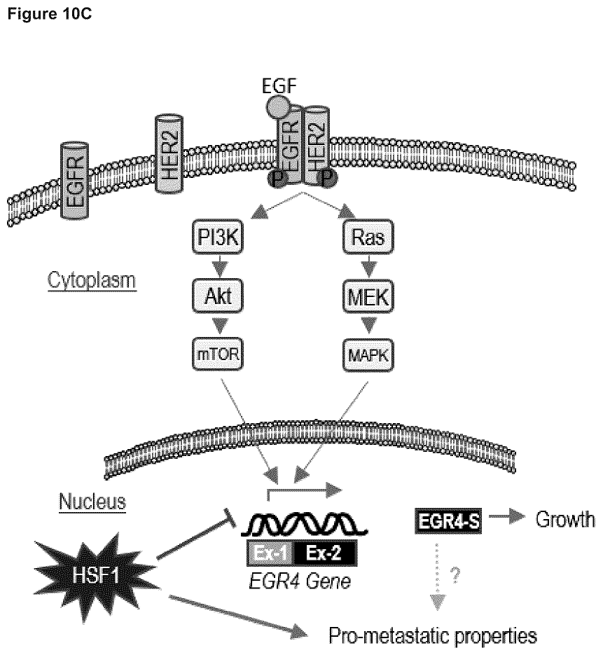

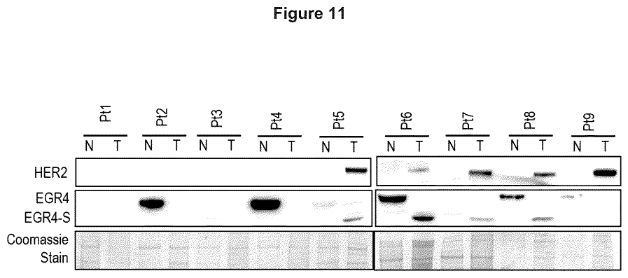

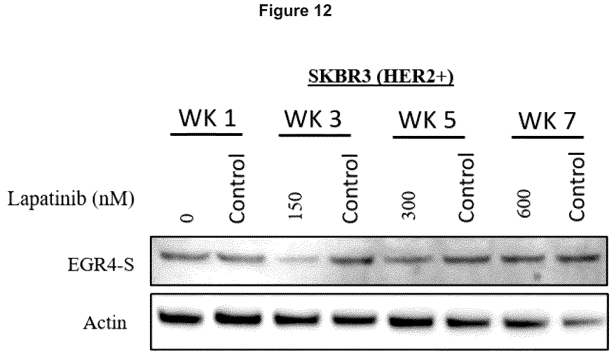

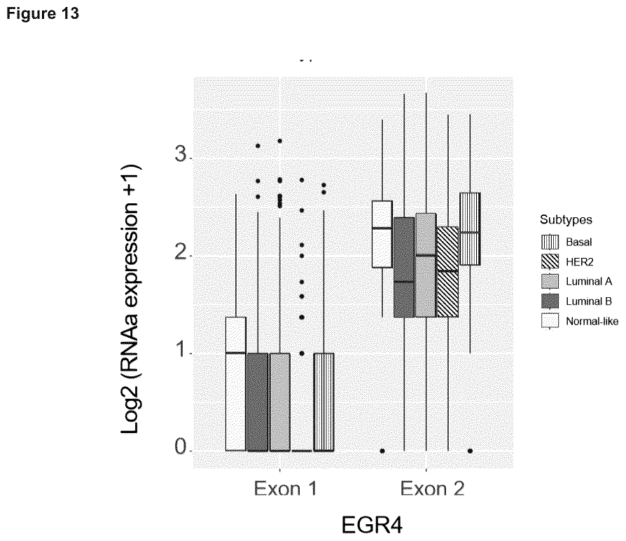

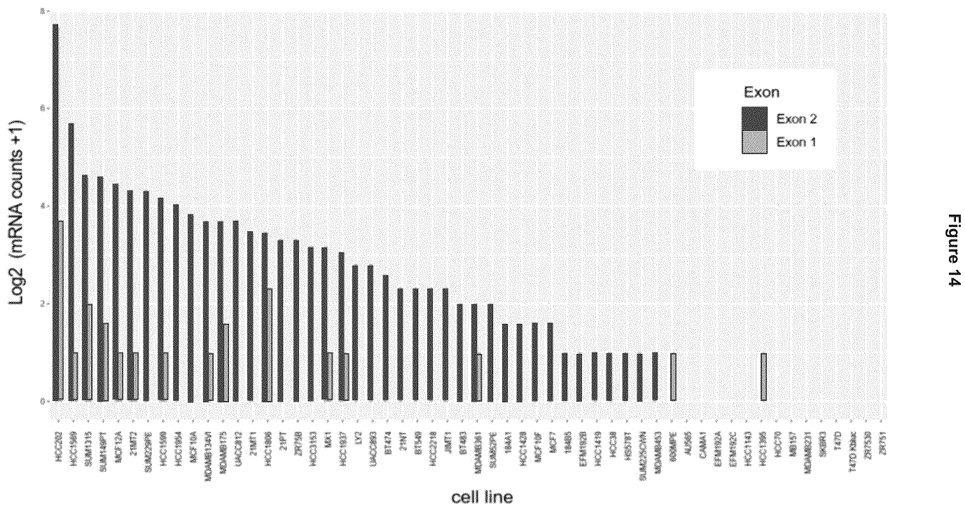

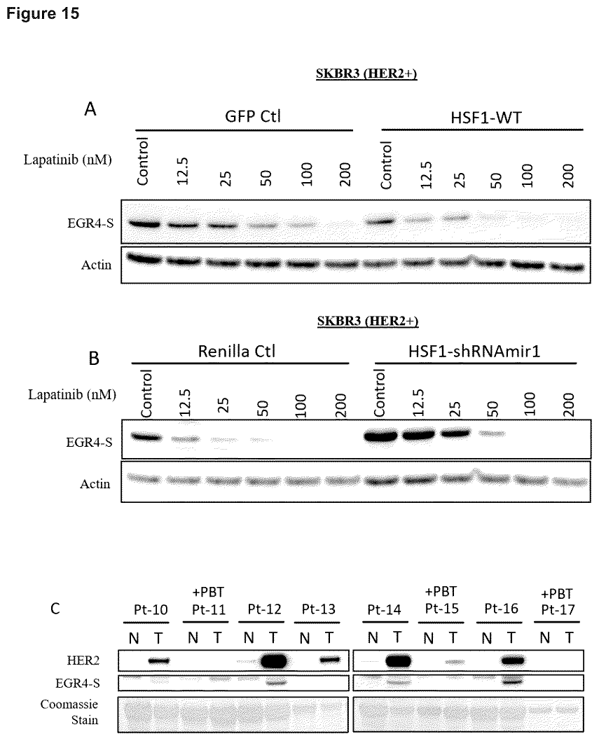

Western blot analysis of these cells (FIG. 1A) confirmed that HSF1 WT, HSF1 ΔRDT and H-Rasv12 were successfully expressed. Increased expression of HSF1 (HSF1 WT or HSF1 ΔRDT) resulted in increased levels of HSPs, such as HSP27 and HSP110 in the cells. Furthermore, ectopic expression of H-Rasv12 altered the morphology of the mammary cells from epithelial to mesenchymal (FIG. 1B). Expression of Ras reduced both the base level and induced expression of HSPs in the cells when combined with HSF1 activation.

FIG. 1A shows that with increased molecular stress resulting from expression of constitutively activated HSF1, oncogenically transformed breast cancer cells (MCF10a cells) had significantly increased expression of HSPs (FIG. 1A) than normal cells expressing constitutively active HSF1.

FIG. 1B shows that with increased molecular stress resulting from expression of constitutively activated HSF1, oncogenically transformed breast cancer cells (MCF10a cells) had a mesenchymal morphology (FIG. 1B) relative to normal cells expressing constitutively active HSF1 which had an epithelial-like morphology.

The glycolytic capacity of the normal and transformed mammary cells was then examined. The results demonstrated that oncogenic transformation with Ras increased the glycolytic capacity of the cells. Increasing HSF1 expression in the transformed cells (HSF1 ΔRDT) resulted in decreased glycolytic capacity (FIG. 3C).

FIG. 1C shows that with increased molecular stress resulting from expression of constitutively activated HSF1, oncogenically transformed breast cancer cells (MCF10a cells) had a significant decrease in glycolytic capacity (FIG. 1C) than normal cells expressing constitutively active HSF1.

### Example 3—EGR4 is Regulated by HSF1 in Normal and Oncogenically Transformed Cells, and EGR4-S Expression Decreases with Increasing HSF1 Expression in Oncogenically Transformed Breast Cells

To investigate the phenomenon of stress decreasing metabolic activity of the cancer cells, microarray analysis was used to compare gene expression in the normal versus oncogenically transformed cells while under the influence of HSF1 ΔRDT. This analysis (FIG. 1D) identified only one gene as being differentially regulated by HSF1 in these two contexts—EGR4.

In normal mammary cells, increased HSF1 resulted in increased expression of EGR4 while, in the cancer cells, increased HSF1 resulted in a decreased expression of EGR4 (FIG. 1E). Upregulation of Ras in the cells increased the level of EGR4.

Unexpectedly, the protein detected in these experiments was not the predicted full-length form of EGR4 (expected to be more than 60 kDa) but a shortened version (51 kDa) referred to herein as “EGR4-S”.

### Example 4— EGR4-S Expression is Increased in Breast Cancer Cell Lines, EGR4-S Expression is Increased HER2+ Breast Cancer, EGR4-S Expression is Decreased in HSF1 Expressing Breast Cancer

Breast cancers are classified into different molecular subtypes based on their gene expression. These include: Luminal A/B, HER2+, Basal and breast-like. EGR4-S expression was examined in a panel of breast cancer cell lines of different subtypes (FIG. 2A).

FIG. 2 demonstrates that EGR4-S is expressed in most of the breast cancer cell lines. Consistent with the finding that EGR4-S expression is upregulated by Ras (FIG. 1E), EGR4-S is highly expressed in cancer cells belonging to the HER2+ group (that have Ras pathway activation).

All cells that expressed high levels of EGR4-S also had lower expression of HSF1 compared to other cell types (FIG. 2A).

### Example 5—EGR4-S is Correlated with HER2+ and Basal Breast Cancer Relapse-Free Survival

To examine the association of EGR4-S with patient survival, kmplot.com (Szasz et al., 2016) was used.

FIG. 2B demonstrates that HER2+ breast cancer patients with high levels of EGR4 expression exhibit a significantly improved relapse-free survival rate.

FIG. 2D demonstrates that Basal breast cancer patients with high levels of EGR4 expression exhibit a significantly improved relapse-free survival rate.

### Example 6—EGR4-S Expression is Responsive to Treatment with Modulators of the HER Signalling Pathway in HER2+ Cells

The effect of modulators that target the HER signalling pathway (TKIs such as Lapatinib, Gefitinib and Erlotinib) on EGR4-S expression was examined.

An analysis of HER2+ cells treated with TKIs showed that, with increasing concentrations of modulator being administered, HER2 phosphorylation and AKT phosphorylation (a downstream molecule in the HER signalling pathway) were both decreased (FIG. 3A).

The reduced expression of EGR4-S observed with increasing drug concentrations mirrored the reduced activity of components of the HER pathway. Importantly, the loss in EGR4-S expression following drug treatment was not transient but maintained over time (FIG. 3B).

Results from similar experiments on EGFR (HER1) expressing breast cancer cells (such as MDA-468) showed that the effect of drug treatment on EGR4-S levels was present but not substantial (FIG. 3C).

This data demonstrates that EGR4-S expression can be reduced by treatment with modulators of the HER signalling pathway.

### Example 7—HSF1 Expression is a Marker of Increased Molecular Stress in Cancer Cells and EGR4-S Expression is a Marker of Decreased Molecular Stress in Cancer Cells

To confirm the link between HSF1 and EGR4-S, the expression of EGR4-S in cancer cells that ectopically expressed HSF1 (HSF1 WT and HSF1 ΔRDT) was examined.

FIG. 4A shows that, in HER2+ breast cells such as MDA-361 and SKBR3, increased levels of HSF1 reduced the expression of EGR4-S breast cancer cells.

Cells identified in previous experiments as having low levels of EGR4-S (such as MCF7 and T47D—see FIG. 2A) exhibited the same inverse relationship to HSF1 expression (FIG. 4B).

FIG. 4C demonstrates that consistent with an inverse relationship between HSF-1 and EGR4-S, knockdown of HSF1 in HS578T basal breast cancer cells (known to have high levels of HSF1) increased the expression of EGR4-S.

FIG. 4D demonstrates that HSF1 expression did not change the total level of HER2, but reduced phosphorylation of the molecule.

FIG. 4E demonstrates that cell stress activating compounds, including “AUY922” and “Sulforaphane” also resulted in the same effect as HSF1 activation by reducing EGR4-S expression.

### Example 8—HSF1 Expression Increased Hallmarks of Metastatic Cancer and HSF1 Expression is a Marker of Metastatic Potential

The cell growth and metastatic potential of HER2+ breast cancer cells were examined over time.

FIG. 5A demonstrates that that overexpression of HSF1 resulted in significantly

However, as per earlier experiments (e.g. FIGS. 6 and 7), FIG. 5C demonstrates that the cancer cells with high levels of HSF1 had significantly greater potential to migrate and had more disorganised growth in 3D culture (FIG. 5B).

### Example 9—Targeted Knockdown of EGR4-S Expression Decreases Cancer Cell Growth

FIG. 5E demonstrates that knockdown of EGR4-S in HER2+ breast cancer cells (demonstrated using Western blot in FIG. 5D) results in significantly reduced cancer cell growth, consistent with the results observed for overexpression of HSF1 in these cells (e.g. FIG. 5A).

### Example 10—EGR4-S is Expressed in Tumour Tissue but not Adjacent Normal Tissue from the Same Patient

The expression of EGR4-S was examined in 9 breast cancer patients, comparing protein expression in tumour tissue and adjacent normal tissue.

FIG. 11 shows a comparison of HER2 and EGR4 expression in HER2− (patients 1-4) and HER2+ (patient 5) breast cancer samples. EGR4-S expression was only detected in the HER2+ sample. Coomassie staining was performed as a protein loading control.

Western blot results from the analysis of HER2 and EGR4 expression in HER2+ breast cancer samples (patients 6-9). EGR4-S was expressed in the majority of the HER2+ breast cancer samples tested, as shown in FIG. 11.

Of patients pre-treated with TKI's to inhibit cancer growth prior to surgery, all (100%) had suppressed EGR4-S expression. Of EGR4-S negative patients receiving treatment, 86% had metastasis or were deceased, whereas of EGR4-S positive patients, 92% had no recurrence, indicating that EGR4-S expression can be used as a marker of metastatic cancer.

### Example 11—Analysis of EGR4 Isoforms

Following characterisation of the different sized EGR, the EGR4 gene was examined. FIG. 8A shows a schematic diagram of the Human EGR4 gene, located on Chromosome 2, showing its 2 exon structure, the 3 possible mRNA transcripts and 3 hypothetical protein isoforms arising from these different transcripts. FIG. 8B shows a biostructural analysis of the 3 protein isoforms of EGR4 reveals 2 potential proteins (EGR4-1 and EGR4-S) and one unlikely possibility (EGR4-2). FIG. 8C shows a schematic diagram of the possible longer EGR4-1 isoform showing two proline-rich regions and multiple phosphorylation sites. FIG. 8D shows a schematic diagram of the shorter/truncated EGR4-S isoform with one single proline-rich region and less phosphorylation sites.

To characterise EGR4-S, 2 sets of primers were designed for different areas of the EGR4 mRNA, as shown in FIG. 9A. qPCR Amplicon 1 (A1) spans the intron/exon boundary whereas PCR Amplicon 2 (A2) sits entirely within exon 2. FIG. 9B shows the details of the primers designed to produce amplicons 1 and 2, along with their binding sites on the full length EGR4 mRNA sequence. The results of the qPCR analysis on breast cancer samples revealed that no product was able to be amplified using Amplicon 1 primers, however Amplicon 2 primers used on the same samples successfully produced a PCR product. In particular, FIG. 9C shows the amplification cycle for qPCR product (mean±SD) detected using primers binding in Amplicon 1 (“EXON 1”) vs primers binding in Amplicon 2/“EXON 2” for the same tumour sample. Amplicon 2/EXON 2 cycle products were detected at a cycle threshold indicating abundant EGR4-S nucleic acid in the samples, and Amplicon 1/EXON 1 cycle products were detected at a cycle threshold indicating no EGR4 nucleic acid in the samples.

### Example 12—EGR4-S Expression is Localised to the Nucleus and Correlates with HER2 Overexpression

FIG. 10A shows representative punch biopsies from a patient with HER2+ breast cancer stained for protein expression, and shows nuclear localisation of EGR4-S and membranous localisation of HER2 in the tumour cells.

FIG. 10B shows HSF1 protein expression is also localised to the nucleus of tumour cells, consistent with it being a transcription factor. An example H&E stain from the samples is presented.

FIG. 10C shows a schematic of the EGFR(HER1)/HER2 signalling pathway and its relationship with the downstream transcription factor EGR4. Activation of the EGFR/HER2 pathway is observed with higher EGR4-S expression and is also correlated with cancer cell growth. Downregulation of EGR4-S expression (e.g. via molecular stress) is correlated with drug resistance and metastasis

FIG. 10D shows that representative punch biopsies from a patient with HER2− breast cancer exhibit no expression of HER2 protein and no nuclear expression of EGR4.

### Example 13—RNA-Seq Data Indicates EGR4-S is Expressed in Basal, HER2, Luminal A, Luminal B and Normal-Like Breast Cancer Subtypes and an Inverse Relationship Between EGR4 Exon 2 Expression and HSF1 Expression

FIG. 13 shows the distribution and median proportional change for expression of EGR4 exon 1 and exon 2 mRNA relative to normal tissue. Expression of mRNA is separated according to breast cancer sub-type. Note that Exon 1 (present only in EGR4, not EGR4-S) was not detected in many samples analysed. FIG. 14 shows EGR4 exon expression in 56 different breast cancer cell lines. Reads were normalised to exon size by scaling to base level coverage. The majority of the cell lines included in the dataset had detectable levels of EGR4 mRNA (n=41 of 56). Of the 56 cell lines, 28 were found to only express exon 2, 13 to express both exons 1 and 2, two cell lines expressed exon 1 alone, and 13 had no detectable expression of EGR4. In cell lines where both exons were detected, the expression of exon 2 was much higher than exon 1 in all cases.

### Example 14—EGR4-S Expression is Responsive to Treatment with a Modulator of the HER Signalling Pathway in HER2+ Cells

The effect of a modulator that targets the HER signalling pathway (Lapatinib) on EGR4-S expression was examined.

Following the observation that EGR4-S expression responds to HER-pathway targeted drug treatment (e.g. Example 6), the effect of modifying cellular expression of HSF1 on a modulator that targets the HER signalling pathway (Lapatinib) on EGR4-S expression was examined.

Lapatinib treatment reduced EGR4-S expression in a dose-dependent manner and this reduction was more pronounced with elevated HSF1 (FIG. 15A).

When HSF1 levels were lowered in the cells (FIG. 15B), EGR4-S expression did not respond as readily to equivalent Lapatinib doses. The cell line was cultured in increasing concentrations of Lapatinib over the course of seven weeks to investigate whether EGR4-S expression would be affected during prolonged treatment with Lapatinib.

After 3 weeks of treatment, reduced levels of EGR4-S could be observed compared to untreated control samples (FIG. 12). However, with prolonged lapatinib treatment, EGR4-S expression was found to be unchanged and expressed at a level comparable to that of untreated control cells. A comparison of HER2 and EGR4-S protein expression on biopsies taken from 8 different breast cancer patients diagnosed with HER2+ tumours (FIG. 15C) showed that no EGR4-S protein could be detected in patients that received pre-biopsy treatment with drugs to suppress the HER2 pathway. The present inventors propose that EGR4-S expression is dependent upon HER2 pathway activation and that EGR4-S may have some value as a biomarker for the inhibition of the HER2 pathway.

1. (canceled)

2. (canceled)

3. A method of identifying a subject with cancer who is likely to be responsive to treatment with a modulator of the HER signalling pathway, wherein the method comprises;
a) providing a sample of the subject;
b) detecting the level of expression of EGR4-S in the sample of the subject, and
c) identifying the subject as being likely to be responsive to treatment with the modulator of the HER signalling pathway if EGR4-S expression is at or above a predetermined level in the sample of the subject, and wherein the subject is likely to be resistant to treatment with the modulator of the HER signalling pathway if EGR4-S expression is at or below a predetermined level in the sample of the subject.

4. The method according to claim 3 further comprising administering to a subject identified as being likely to be responsive to treatment with the modulator of the HER signalling pathway a therapeutically effective amount of the modulator of the HER signalling pathway.

5. (canceled)

6. The method according to claim 3, further comprising detecting the level of expression of HSF1 in the sample of the subject.

7. The method according to claim 6, wherein the subject is likely to be resistant to treatment with the modulator of the HER signalling pathway if EGR4-S expression is at or below a predetermined level in the sample of the subject, and HSF1 expression is at or above a predetermined level in the sample of the subject.

8. The method according to claim 6, wherein the subject is likely to be responsive to treatment with the modulator of the HER signalling pathway if EGR4-S expression is at or above a predetermined level in the sample of the subject, and HSF1 expression is at or below a predetermined level in the sample of the subject.

9. The method according to claim 3, wherein the method further comprises detecting the level of expression of HER1 and/or HER2 in a sample of the subject.

10. The method according to claim 9, wherein the subject is likely to be responsive to treatment with the modulator of the HER signalling pathway if EGR4-S, and HER1 and/or HER2, is at or above a predetermined level in the sample of the subject.

11. The method according to claim 10, wherein the predetermined level is the amount or level of EGR4-S, HSF1, HER1 and/or HER2 in a control or reference sample.

12. (canceled)

13. The method according to claim 3 wherein the modulator of the HER signalling pathway is an EGFR inhibitor.

14. A method according to claim 3 wherein the step of detecting the level of expression of EGR4-S in the sample comprises detecting EGR4-S mRNA expression.

15. The method according to claim 3 wherein the step of detecting the level of expression of EGR4-S in the sample comprises detecting EGR4-S nucleic acid expression and/or EGR4-S polypeptide expression.

16-18. (canceled)

19. The method of claim 3 wherein the cancer is a solid tumour.

20. The method according to claim 19 wherein the solid tumour is breast cancer tumour or lung cancer.

21-24. (canceled)

25. A method of treating or preventing a HER pathway activated cancer; wherein the method comprises administering to a subject a therapeutically effective amount of a modulator of EGR4-S activity.

26. (canceled)

27. The method according to claim 25 wherein the modulator of EGR4-S activity is selected from the group consisting of an EGFR inhibitor, an siRNA, and a shRNA.

28-32. (canceled)

34. A method of detecting EGR4-S in a subject, said method comprising
a) providing a sample of the subject;
b) detecting the presence of EGR4-S in the sample of the subject by a method comprising detecting EGR4-S polypeptide expression and/or EGR4-S nucleotide expression.

35. The method according to claim 34 wherein step b) comprises a method comprising using PCR, RT-PCR across the EGR4 exon 1/exon 2 splice site, FISH, an immunohistochemical assay for EGR4-S polypeptide expression, histology, ELISA, an ELISA-like assay, Western Blot, and/or flow cytometry to assay for EGR4-S polypeptide expression.
